(19) United States
(12) Patent Application Publication
(10) Pub. No.: US 2026/0347853 A1
(43) Pub. Date: Nov. 5, 2026

(54) PREFERRED CLEAVAGE MOTIF OF THE 10-23 DNA ENZYME

(71) Applicant: THE REGENTS OF THE UNIVERSITY OF CALIFORNIA, OAKLAND, CA (US)

(72) Inventors: John C. Chaput, IRVINE, CA (US); Kim T. Nguyen, IRVINE, CA (US); Turnee N. Malik, IRVINE, CA (US)

(73) Assignee: THE REGENTS OF THE UNIVERSITY OF CALIFORNIA, OAKLAND, CA (US)

(21) Appl. No.: 19/134,679

(22) PCT Filed: Dec. 1, 2023
(86) PCT No.: PCT/US2023/082198

§ 371 Date: May 30, 2025

Related U.S. Application Data
(60) Provisional application No. 63/429,504, filed on Dec. 1, 2022.

(51) Int. Cl. C12N 15/113

(52) U.S. Cl. CPC C12N 15/1135; C12N 2310/127; C12N 2310/321; C12N 2310/3231; C12N 2310/344; C12N 2310/3517; C12N 2320/34; C12N 2320/50

(57) ABSTRACT

Described herein are new modified 10-23 DNAzymes and related methods for gene silencing. The modified DNAzyme functions with unparalleled catalytic activity under physiological conditions. For the last 25-years, pharmaceutical companies interested in developing 10-23 DNAzymes have determined the best reagents by designing, constructing, and testing 10-23 DNAzymes against all of the G-U dinucleotides in a given mRNA target, a process that is both expensive and laborious. The approach described herein streamlines the drug discovery process by considering the nucleotide positions adjacent to the G-U cut site. Specifically, UGUD motifs (G-U cut site is underlined) function with the highest catalytic activity, meaning that optimal 10-23 DNAzymes will have an A at position 16 and an A or Y at position-2, where Y is the universal symbol for a pyrimidine (C or T).

This application claims benefit of U.S. provisional patent application No. 63/429,504, filed Dec. 1, 2022, the entire contents of which are incorporated by reference into this application.

### REFERENCE TO A SEQUENCE LISTING SUBMITTED

The content of the XML file of the sequence listing named “UCI015_Seq” which is 141 kb in size was created on Jun. 5, 2026, and electronically submitted herewith the application, is incorporated herein by reference in its entirety.

### BACKGROUND

RNA-cleaving DNA enzymes (DNAzymes or Dz), such as Dz 10-23, offer an attractive modality for targeting undruggable regions of the human genome (1,2). These reagents benefit from inexpensive and scalable protocols for manufacturing designer sequences by solid-phase chemical synthesis, programmable binding arms that can be engineered to recognize nearly any RNA target, a highly specific cleavage mechanism that dramatically reduces activity against non-homologous sequences, and high safety and tolerability margins in human patients (3,4). However, despite their many advantages, interest in DNAzymes as gene silencing agents in therapeutic applications has declined due to their low efficacy in clinical trials. This problem, which is primarily due to their poor catalytic activity under physiological conditions, raises an outstanding challenge in the field of nucleic acid chemistry of how to develop DNAzymes that can maintain high catalytic activity in cellular environments where the concentration of free magnesium (Mg2+) is limiting (5,6).

The last 30 years have witnessed tremendous growth in the evolution of chemical modifications for oligonucleotide therapeutics. Although antisense oligonucleotides (ASOs) and small interfering RNAs (siRNA) were the primary focus of this effort (7,8), chemical modifications have also improved the stability and catalytic activity of DNAzymes (9). Modifications commonly observed in DNAzyme scaffolds include the addition of inverted 3′-3′ thymidine nucleotides 10, the substitution of phosphodiester linkages for phosphorothioate (11) or phosphoramidite linkages (12), and the replacement of natural DNA residues with unnatural xeno-nucleic acid (XNA) residues (13), such as 2′-O-methylribonucleic acid (OMe) (14), locked nucleic acid (LNA) (15), and 2′-fluoroarabinonucleic acid (FANA) (16). Knowledge gained from these studies led to the development of new DNAzyme designs that function with improved activity, including a recent X10-23 design that exhibits substantial (>60%) reductions of mRNA and protein levels in cells (17). Elsewhere in the field, polymerase engineering has enabled the evolution of RNA-cleaving XNA enzymes (XNAzymes) composed entirely of non-natural nucleotides (18,19,20,21). While these studies have narrowed the gap between DNAzymes and protein-based gene silencing agents, the ability to routinely generate DNAzymes with robust multiple turnover activity under physiological conditions remains an unsolved problem.

There remains a need for DNAzymes with substantial catalytic activity under physiological conditions, and for understanding the preferred motif for optimal cleavage in order to select the best target sites in an RNA sequence.

### SUMMARY

The materials and methods described herein meet these needs and more by providing a DNAzyme that has been engineered to target the preferred sequence motif required for optimal cleavage of an RNA sequence. As described herein, structural constraints imposed by chemical modifications drive catalysis toward a highly preferred UGUD motif (cut site underlined; D is U/R or U/A/G) that has been validated across several mRNA targets This DNAzyme provides a tool for precision allele-specific gene silencing, as well as diagnostic and therapeutic applications.

Described herein is a nucleotide construct comprising: a 3′ substrate binding arm; a 5′ substrate binding arm and a catalytic domain. The catalytic domain has the nucleic acid sequence of positions 1-15 of SEQ ID NO: 1, and is disposed between the 3′ substrate binding arm and the 5′ substrate binding arm. The 3′ substrate binding arm and the 5′ substrate binding arm are complementary to nucleotides flanking a G-U cleavage site in a target mRNA, wherein the first nucleotide 3′ of the catalytic domain is an adenosine (A), and wherein the second nucleotide 5′ of the catalytic domain is an A or a pyrimidine (Y). The sequence of the nucleotide construct is thus targeted to a G-U cleavage site, which cleavage site is a U-G-U-U or a U-G-U-R sequence. R, as is understood in this context, refers to a purine nucleotide. A G-U cleavage site in the context of such a 5′-UGUU/R-3′ (or 5′-UGUD-3′) motif is up to 5-fold more active than 5′-XGUU-3′ motifs, where X corresponds to a G, A, or C residue.

In some embodiments, the substrate binding arms comprise one or more modified nucleic acids. Such modified nucleic acids can be at any position within the substrate binding arms. In some embodiments, a first modified nucleic acid is disposed at the terminus of the 3′ substrate binding arm, a second modified nucleic acid is disposed at the terminus of the 5′ substrate binding arm, and three OMe modifications are positioned adjacent to each of the modified nucleic acids.

In some embodiments, the modified nucleic acids comprise a MOE, locked nucleic acid (LNA), threose nucleic acid (TNA), mannitol nucleic acid (MNA), serinol nucleic acid (SNA), glycol nucleic acid (GNA), hexitol nucleic acid (HNA), diaminopurine (DAP), C-5 modified pyrimidine, pseudo uridicine, or C-5-methyl deoxycytidine. In some embodiments, the modified nucleic acid is an LNA or MOE. MOE is an equivalent, if not slightly better, modification at the termini. MOE is expected to be less toxic than LNA.

In some embodiments, the 3′ substrate binding arm and the 5′ substrate binding arm are each at least 5 nucleotides in length. In some embodiments, the 3′ substrate binding arm and the 5′ substrate binding arm are each 6 to 10 nucleotides in length. In some embodiments, the substrate binding arms are each about 8 nucleotides in length. In some embodiments, the 3′ substrate binding arm and the 5′ substrate binding arm are complementary to nucleotides flanking a G-U cleavage site in a target RNA.

In some embodiments, the target RNA is Kirsten Rat Sarcoma Viral Oncogene Homolog (KRAS) mRNA.

In some embodiments, the dC at position 7 and the dT at position 8 are each substituted with a 2′-O-methylribonucleic acid (OMe) residue to form an OMe-C and an OMe-U, respectively. In addition, the G at position 14 is substituted with an OMe or a 2′-O-methoxyethylribonucleic acid (MOE) residue to form an OMe-G or an MOE-G, and a first phosphorothioate linkage is disposed between positions 0 and 1. In some embodiments, the G at position 14 is an MOE-G. This MOE-G at position 14 embodiment exhibits the fastest turnover rate under physiological conditions. In some embodiments, the nucleotide construct further comprises a second phosphorothioate linkage between the dC at position 13 and the dG at position 14.

Also described is a method of cleaving a target RNA sequence. In some embodiments, the method comprises contacting the target RNA sequence with a nucleotide construct as described herein. Representative examples of target RNA include, but are not limited to, mRNA and viral RNA. In some embodiments, the 3′ substrate binding arm and the 5′ substrate binding arm are complementary to nucleotides flanking a G-U cleavage site in Kirsten Rat Sarcoma Viral Oncogene Homolog (KRAS) mRNA. Other examples of a target RNA include, but are not limited to, viral or bacterial sequences, such as sequences specific to SARS-COV-2 or other pathogens, including particular strains or subtypes of interest.

Additionally described is a method of modulating the translation of a disease-associated protein. Modulating, or disrupting, the translation of the disease-associated protein has the effect of silencing the corresponding gene. In some embodiments, the method comprises contacting a target mRNA sequence that encodes the disease-associated protein with a nucleotide construct as described herein. Examples of disease-associated proteins include, but are not limited to, cancer-related proteins.

Also provided is a method of treating a disease or condition or ameliorating a symptom thereof. In some embodiments, the method comprises administering an effective amount of a nucleotide construct as described herein to a subject in need thereof. In some embodiments, the disease or condition is caused by a genetic disease, viral or bacterial pathogen, cancer, inflammation, cardiovascular disease, immune deficiency, or a neurological disorder. In some embodiments, the cancer is associated with KRAS mutations. In some embodiments, the disease or condition is lung, pancreatic, or colorectal adenocarcinoma, melanoma.

Further provided is a method of detecting the presence of a target RNA sequence in a sample. In some embodiments, the method comprises contacting the sample with a nucleotide construct as described herein, wherein the 3′ substrate binding arm and the 5′ substrate binding arm of the nucleotide construct are complementary to the target RNA sequence. The method further comprises measuring cleavage of the target RNA sequence, wherein cleavage is indicative of presence of the target RNA sequence in the sample.

In some embodiments, the nucleotide construct is modified with a marker that is detectable upon cleavage, and wherein the measuring comprises measuring the presence of the detectable marker. In some embodiments, the detectable marker is a fluorophore. For example, a quencher may be positioned in the construct such that, upon cleavage, the fluorophore is no longer subjected to quenching, thereby resulting in detectable signal. A representative example of a detection method that employs such a DNAzyme is described in Yang et al., 2022, Journal of the American Chemical Society 2022 144 (26), 11685-11692 DOI: 10.1021/jacs.2c03420. The SARS-COV-2 region of interest can be isothermally amplified by RT-RPA and T7 RNA polymerase to produce multiple copies of the RNA analyte, or optionally, detection can be achieved without pre-amplification. Competitive DNAzyme assembly on the viral RNA produces a fluorescent signal specific to the sample genotype via site-specific cleavage of a quenched fluorescent reporter.

### BRIEF DESCRIPTION OF THE DRAWINGS

FIG. 1 provides the nucleotide sequence of the catalytic domain of 10-23 and adjacent nucleotides of the substrate binding arm (SEQ ID NO: 22) and its complementarity to an RNA substrate having a UGUU/R cleavage site.

FIGS. 2A-2E illustrate the chemical evolution of DNAzyme 10-23. (2A) Cartoon representation of an NMR-averaged precatalytic structure of 10-23 bound to an RNA substrate (marked with dot in center) encoding a prion protein (“7PDU”). Dz is shown in gray, with numbers matching residues found to be important in the chemically optimized version. (2B) Left: Dz 10-23_v46 (SEQ ID NO: 2) in complex with a 16-nt KRAS G12V RNA substrate (upper sequence, SEQ ID NO: 4). Right: Chemical structures of natural and modified nucleotides found in Dz 10-23_v46. Modifications are indicated, which correspond to positions in Dz 10-23 as follows: LNA at the position at each end, OMe at the next 3 positions internal to the LNAs at each end as well as at C7 and U8, and MOE at G14. PS indicates phosphorothioate linkages. (2C) Representative denaturing PAGE gels showing time-dependent multiple turnover RNA cleavage activity across a panel of engineered Dz 10-23 variants. S: 5′-Cy5-labeled full-length substrate (16 nt), P: 5′-Cy5-labeled cleavage product. (2D, 2E) Initial velocities (vₒ, 2D) and kinetic curves (2E) were measured for each variant. Error bars denote the ═standard deviation of the mean for three independent replicates. All reactions were performed under simulated physiological conditions in a buffer containing 1 mM MgCl₂, 50 mM Tris (pH 7.5), 10 mM NaCl, and 140 mM KCl at 37 °C with 1000 nM substrate and 10 nM enzyme (100:1, S:E).

FIGS. 3A-3C illustrates the nearest neighbor analysis uncovers an expanded GU cleavage motif. (3A) Engineered Dz 10-23_v46 version (SEQ ID NO: 2, 5-10, respectively, top to bottom at left, then right column) and matching substrate designs (SEQ ID NO: 4, 11-16, respectively). Nucleotide positions immediately flanking the 5′ and 3′ sides of the GU dinucleotide cleavage junction were individually varied (boxes) to identify a preferred substrate cleavage motif. The modifications for DNAzyme-substrate pairs is the same as shown in FIG. 2B. (3B) Normalized initial rates observed for each DNAzyme-substrate pair reveal a strong preference for 5′-UGUU-3′ and 5′-UGUR-3′ cleavage sites, where R refers to a purine. (3C) Positive (top) and negative (bottom) predictions based on the preferred 5′-UGUU/R-3′ cleavage motifs. Known GATA3 and c-jun cleavage sites function with inferior activity as compared to their engineered DNAzyme-substrate pairs containing the preferred UGUU motif. Previously untargeted HTT and PCSK9 cleavage sites function with superior activity relative to their low-activity motifs. Mutated nucleotides in the substrate are first and last U of UGUU for GATA3, first U of UGUU for c-jun, C of CGUU for HTT, and A of AGUU for PCSK9. Error bars denote the standard deviation of the mean for two independent replicates. Reactions were performed in a buffer containing 1 mM MgCl₂, 50 mM Tris (pH 7.5), 10 mM NaCl, and 140 mM KCl at 37 °C with 1000 nM substrate and 10 nM enzyme (100:1, S:E).

FIGS. 4A-4D illustrate autonomous DNAzyme-mediated RNA cleavage mechanism. (4A) Reagents used to evaluate the mechanism of RNA cleavage include a 60 nt RNA segment of KRAS G12V (SEQ ID NO: 17), Dz 10-23_v46 (active Dz; SEQ ID NO: 2; corresponding substrate SEQ ID NO: 4), Dz 10-23_v47 (inactive Dz; SEQ ID NO: 18; corresponding substrate SEQ ID NO: 4), and a 22 nt linear DNA (SEQ ID NO: 20; corresponding 22nt substrate SEQ ID NO: 19). The DNAzyme recognition site on the RNA substrate is at positions 23-38, and the RNA cleavage site is indicated with an arrow. The modifications for DNAzyme-substrate pairs is the same as shown in FIG. 2B. (4B) Representative denaturing PAGE gels showing RNA cleavage profiles in the presence (+) or absence (−) of E. coli RNase HI (0.05 U/μL) after 0.5, 5, and 30 min of incubation (Dz 46 and 47, n=3; 48, n=1). (4C) Quantification of RNase H1 in three mammalian cell lines by ELISA. Cellular levels of RNAse H1 range from 0.5 to 2.5 ng/μL. Error bars denote the ±standard deviation of the mean for three independent replicates. (4D) Denaturing PAGE gel showing RNA cleavage profiles in the absence (−) or presence (+) of human RNase H1 after a 30 min incubation (n=1). Linear DNA is a positive control. Reactions were performed in a buffer containing 1 mM MgCl₂, 50 mM Tris (pH 7.5), 10 mM NaCl, and 140 mM KCl at 37 °C with 250 nM substrate and 250 nM enzyme (1:1, S:E). S: 5′-Cy5-labeled full-length substrate, P: 5′-Cy5-labeled cleavage product.

FIGS. 5A-5C illustrate allele-specific targeting of wild-type and KRAS G12V RNA under cell-free conditions. (5A) Substrates used to evaluate allele-specific RNA cleavage of KRAS G12V include two 60 nt segments of KRAS mRNA that differ by a single point mutation (SEQ ID NO: 17, 21). The G12V version (SEQ ID NO: 17) carries a G-to-U mutation (shaded) that leads to a glycine-to-valine mutation in the translated KRAS protein. RNA binding sites targeted by the wild-type (49) and G12V (46)-specific DNAzymes of Dz 10-23_v46 are at positions 23-38 for G12V and positions 24-39 for wild-type, with the cleavage site indicated with an arrow. (5B) Denaturing PAGE gel showing allele-specific RNA cleavage of both alleles by the wild-type (49) and G12V (46)-specific DNAzymes after 5 and 30 min (n=3). Reactions were performed in a buffer containing 1 mM MgCl₂, 50 mM Tris (pH 7.5), 10 mM NaCl, and 140 mM KCl at 37 °C with 500 nM substrate and 50 nM enzyme (10:1, S:E). S: 5′-Cy5-labeled full-length substrate, P: 5′-Cy5-labeled cleavage product. (5C) Denaturing PAGE gel showing RNA cleavage profiles in reactions containing both the wild-type (red) and G12V (green) substrates (1:1) in the absence (−) or presence (+) of 5 ng/μL human RNase H1 after a 30-min incubation (n=1). Individual Cy5 (red) and AlexaFluor750 (green) channels, as well as the merged image, are shown. Reactions were performed using unpaired Dz (50), inactive Dz (47), active G12V Dz (46), and 22 nt linear DNA control (48) in buffer containing 1 mM MgCl₂, 50 mM Tris (pH 7.5), 10 mM NaCl, and 140 mM KCl at 37 °C with 250 nM total substrate and 250 nM enzyme.

FIGS. 6A-6C illustrate allele-specific targeting of endogenous KRAS G12V RNA in a lung cancer cell model. (6A) Schematic representation of the transcription and translation of allelic mutations associated with the expression of wild-type and G12V mutant KRAS proteins in NCI-H441 cells. (6B) Schematic view of the PCR restriction fragment length polymorphism assay (PCR-RFLP) used to distinguish KRAS alleles associated with wild-type (SEQ ID NO: 23) and mutant G12V (SEQ ID NO: 4) protein expression. G-to-C mutation introduced by primer during PCR is shown in bold at 5th position. The second position of codon 12 (boxed) encoding G12V (SEQ ID NO: 4) or WT (SEQ ID NO: 23) KRAS is shown in bold. Converted sequences correspond to SEQ ID NO: 24, 25 (WT) and 26, 27 (G12V). (6C) Quantification of allele-specific G12V knockdown levels observed in NCI-H441 cells after 6, 12, and 24 h of treatment with either 50 or 500 nM Dz 10-23_v46 targeting KRAS G12V mRNA. Representative PCR-RFLP results are provided as an insert for each plot (n=4). Error bars denote #standard deviation of the mean for n=4:2 biological and 2 technical replicates.

FIG. 7 provides structural views of DNAzyme 10-23. Cartoon representation of an NMR-averaged precatalytic structure of 10-23 bound to an RNA substrate (dot at center of substrate) encoding a prion protein (PDB: 7PDU). Structural views observed at 120° and 240° horizonal rotations and vertical rotations of 180°.

FIG. 8 illustrates mechanism of DNAzyme-mediated RNA cleavage. Site-specific RNA cleavage occurs by deprotonation of the 2′ hydroxyl group of the scissile residue with simultaneous protonation of the 5′ hydroxyl group of the cleaved bond. In-line attack of the resulting 2′ oxyanion on the adjacent phosphate results in the formation of an upstream cleavage product carrying cyclic phosphate and downstream cleavage product with a hydroxyl group.

FIGS. 9A-9C demonstrates the structure activity relationship analysis of position 14 in the catalytic loop of DNAzyme 10-23. (9A) Nucleic acid sequence of DNAzyme 10-23 (SEQ ID NO: 28) targeting a segment of KRAS G12V mRNA (SEQ ID NO: 4) and chemical structures of sugar modified analogs. (9B, 9C) Representative gel and bar graph showing the cleavage activity of each analog (n=1). All reactions were performed in simulated physiological buffer containing 1 mM MgCl₂, 50 mM Tris (pH 7.5), 10 mM NaCl, and 140 mM KCl at 37 °C with 500 nM substrate and 50 nM enzyme (10:1, S:E). S: 5′-Cy5 labeled full-length substrate, P: 5′-Cy5 labeled cleavage product. Molecular weight markers indicated to the right of the gel.

FIGS. 10A-10C show the structure activity relationship analysis of 10-23 variants prepared with modified binding arms, G14 modification and a phosphodiester linkage at position 0-1 of the catalytic loop. (10A) Sequences of DNAzyme 10-23 (wild-type, SEQ ID NO: 29, and engineered variants: (10) with 3 LNAs at each of the 3′ and 5′ ends and an OMe at G14, SEQ ID NO: 30, (13) with one LNA at each of the 3′ and 5′ ends, and OMe at the 3 positions internal to the LNA of each substrate binding arm as well as at G14, SEQ ID NO: 31) targeting a segment of KRAS G12V mRNA (SEQ ID NO: 4) and chemical structures of sugar modified analogs. Abbreviations of nucleotide analogs: LNA (locked nucleic acid), OMe (2′-O-methylribonucleic acid), and phosphorothioate. (10B, 10C) Representative gels and bar graph showing the cleavage activity of each analog (n=1). All reactions were performed in simulated physiological buffer containing 1 mM MgCl₂, 50 mM Tris (pH 7.5), 10 mM NaCl, and 140 mM KCl at 37 °C with 500 nM substrate and 50 nM enzyme (10:1, S:E). S: 5′-Cy5 labeled full-length substrate, P: 5′-Cy5 labeled cleavage product. Molecular weight markers indicated to the right of the gel.

FIG. 11 shows results of a 2′-methoxy walk of the catalytic loop for double modifications that enhance activity. Sequence of DNAzyme 10-23 (variant 10; SEQ ID NO: 30) targeting a segment of KRAS G12V mRNA (SEQ ID NO: 4) and chemical structures of sugar modified analogs. Bar graph denotes the cleavage activity of each 10-23 variant after a 5 minute incubation at 37 °C (n=1). Color scheme: 10-23 variant 9 (black), 10-23 variant 10 (dark gray), and 10-23 variant 10 modified with a second OMe residue in the catalytic loop (gray). All reactions were performed in simulated physiological buffer containing 1 mM MgCl₂, 50 mM Tris (pH 7.5), 10 mM NaCl, and 140 mM KCl with 500 nM substrate and 50 nM enzyme (10:1, S:E). S: 5′-Cy5 labeled full-length substrate, P: 5′-Cy5 labeled cleavage product.

FIGS. 12A-12C show the structure-activity analysis of positions 7, 8, and 14 in the catalytic loop with 2′-methoxy residues. (12A) Sequence of DNAzyme 10-23 (SEQ ID NO: 32) targeting a KRAS G12V mRNA segment (SEQ ID NO: 4) and chemical structures of sugar modified analogs. Abbreviation: OMe (2′-O-methylribonucleic acid). (12B, 12C) Representative gels and bar graph showing the cleavage activity of each analog (n=1). All reactions were performed in simulated physiological buffer containing 1 mM MgCl₂, 50 mM Tris (pH 7.5), 10 mM NaCl, and 140 mM KCl at 37 °C with 500 nM substrate and 50 nM enzyme (10:1, S:E). S: 5′-Cy5 labeled full-length substrate, P: 5′-Cy5 labeled cleavage product. Molecular weight markers indicated to the right of the gel.

FIGS. 13A-13C show the structure-activity analysis of positions 7, 8, and 14 in the catalytic loop with 2′-methoxyethoxy residues. (13A) Sequence of DNAzyme 10-23 (SEQ ID NO: 33) targeting a KRAS G12V mRNA segment (SEQ ID NO: 4) and chemical structures of sugar modified analogs. (13B, 13C) Representative gels and bar graphs showing the cleavage activity of each analog (n=1). All reactions were performed in simulated physiological buffer containing 1 mM MgCl₂, 50 mM Tris (pH 7.5), 10 mM NaCl, and 140 mM KCl at 37 °C with 500 nM substrate and 50 nM enzyme (10:1, S:E). S: 5′-Cy5 labeled full-length substrate, P: 5′-Cy5 labeled cleavage product. Molecular weight markers indicated to the right of the gel.

FIGS. 14A-14C show the structure-activity analysis of positions 7, 8, and 14 with a phosphorothioate linkage at position 0-1 in the catalytic loop. (14A) Sequence of DNAzyme 10-15 (SEQ ID NO: 34) targeting a KRAS G12V mRNA segment (SEQ ID NO: 4) and chemical structures of sugar modified analogs. (14B, 14C) Representative gels and bar graph showing the cleavage activity of each analog (n=1). All reactions were performed in simulated physiological buffer containing 1 mM MgCl₂, 50 mM Tris (pH 7.5), 10 mM NaCl, and 140 mM KCl at 37 °C with 500 nM substrate and 50 nM enzyme (10:1, S:E). S: 5′-Cy5 labeled full-length substrate, P: 5′-Cy5 labeled cleavage product. Molecular weight markers indicated to the right of the gel.

FIGS. 15A-15C illustrate chemical optimization of 10-23 variant 13. (15A) Sequence of DNAzyme 10-23 (SEQ ID NO: 31) targeting a segment of KRAS G12V mRNA (SEQ ID NO: 4) and chemical structures of sugar modified analogs. (15B, 15C) Representative gel and bar graph showing the cleavage activity of each analog (n=1). All reactions were performed in simulated physiological buffer containing 1 mM MgCl₂, 50 mM Tris (pH 7.5), 10 mM NaCl, and 140 mM KCl at 37 °C with 500 nM substrate and 50 nM enzyme (10:1, S:E). S: 5′-Cy5 labeled full-length substrate, P: 5′-Cy5 labeled cleavage product. Molecular weight markers indicated to the right of the gel.

FIGS. 16A-16C illustrate chemical optimization of 10-23 variant 42 measured at 100:1 (S:E). (16A) Sequence of DNAzyme 10-23 (SEQ ID NO: 35) targeting a segment of KRAS G12V mRNA (SEQ ID NO: 4) and chemical structures of sugar modified analogs. (16B-16C) Representative gels and bar graphs showing the cleavage activity of each analog (n=1). All reactions were performed in simulated physiological buffer containing 1 mM MgCl₂, 50 mM Tris (pH 7.5), 10 mM NaCl, and 140 mM KCl at 37 °C with 1000 nM substrate and 10 nM enzyme (100:1, S:E). S: 5′-Cy5 labeled full-length substrate, P: 5′-Cy5 labeled cleavage product. Molecular weight markers indicated to the right of the gel.

FIG. 17 provides a summary of 10-23 Designs Evaluated in Kinetic Cleavage Assays. Sequence of 10-23 DNAzyme variants SEQ ID NOS: 29, 36, 37, 38, 31, 39, 40, 35, 2, respectively) targeting a size-matched segment of KRAS G12V mRNA (SEQ ID NO: 4) and chemical structures of sugar modified analogs. G14 is an OMe in variants (3), (13), (31), and (38). G14 is an MOE in variants (4), (42), and (46). C7 and U8 are OMe in variants (31), (38), (42), and (46).

FIGS. 18A-18C illustrate multiple turnover kinetic analysis of 10-23 variants measured at 10:1 substrate to enzyme concentration. (18A) Representative denaturing PAGE gels of timedependent RNA cleavage assays. (18B-18C) Kinetic curves plotted after (18B) and 30 (18C) minutes. All reactions were performed in simulated physiological buffer containing 1 mM MgCl₂, 50 mM Tris (pH 7.5), 10 mM NaCl, and 140 mM KCl at 37 °C with 500 nM substrate and 50 nM enzyme (10:1, S:E). S: 5′-Cy5 labeled full-length substrate, P: 5′-Cy5 labeled cleavage product. Molecular weight markers indicated to the right of the gel. Error bars denote±standard deviation from the mean of 3 independent replicates.

FIG. 19 graphically depicts the transferability of Dz 46 chemistry to other targets. Initial velocities (v0) and kinetic curves measured for Dz 1 (lower) and Dz 46 (upper) versions of PCSK9, HTT, GATA3, and c-jun sequences. All four targets utilize the UGUU cleavage motif evaluated in FIG. 3C. Reported values represent the mean for 2 independent replicates. The initial velocity for Dz 1 targeting HTT could not be determined due to a lack of cleavage activity observed under the 60 min timeframe. All reactions were performed under simulated physiological conditions in buffer containing 1 mM MgCl₂, 50 mM Tris (pH 7.5), 10 mM NaCl, and 140 mM KCl at 37 °C with 1000 nM substrate and 10 nM enzyme (100:1, S:E).

FIGS. 20A-20C demonstrate the multiple turnover kinetic analysis comparing variant 46 to known methoxy derivatives at 100:1 substrate to enzyme concentration. (20A) Nucleic acid sequence of chemically engineered 10-23 variants (SEQ ID NOS: 2, 41, 42, respectively; all bold positions in DV15E4 and DH5E are OMe) targeting a segment of KRAS G12V RNA (SEQ ID NO: 4). (20B) Representative denaturing PAGE gels of time-dependent RNA cleavage assays. (20C) Kinetic curves plotted over a reaction time of 30 minutes. All reactions were performed in simulated physiological buffer containing 1 mM MgCl₂, 50 mM Tris (pH 7.5), 10 mM NaCl, and 140 mM KCl at 37 °C with 1000 nM substrate and 10 nM enzyme (100:1, S:E). S: 5′-Cy5 labeled full-length substrate, P: 5′-Cy5 labeled cleavage product. Molecular weight markers indicated to the right of the gel. Error bars denote ±standard deviation from the mean of 3 independent replicates.

FIGS. 21A-21D shows the magnesium dependency of engineered 10-23 variants measured at 100:1 substrate to enzyme concentration. (21A) Nucleic acid sequence of 10-23 (SEQ ID NO: 29) and chemically engineered 10-23 variants (SEQ ID NOS: 40, 35, 2, respectively) targeting a segment of KRAS G12V RNA substrate (SEQ ID NO: 4). (21B) Chemical structures of sugar modified analogs. (21C, 21D) Representative gels and bar graphs showing the cleavage activity of 10-23 variants after 20 minutes of incubation at defined concentrations of MgCl₂. All reactions were performed in simulated physiological buffer containing 50 mM Tris (pH 7.5), 10 mM NaCl, and 140 mM KCl at 37 °C with 1000 nM substrate and 10 nM enzyme (100:1, S:E). S: 5′-Cy5 labeled full-length substrate, P: 5′-Cy5 labeled cleavage product. Molecular weight markers indicated to the right of the gel. Error bars denote±standard deviation from the mean of 3 independent replicates.

FIGS. 22A-22C demonstrate the Activity of Dz 46 under reduced concentrations of magnesium. (22A) Bar graph showing normalized initial rates for Dz 46 in the presence or absence of 1 mM ATP. Reactions were performed in simulated physiological buffer containing 1 mM MgCl₂, 50 mM Tris (pH 7.5), 10 mM NaCl, and 140 mM KCl at 37 °C with 1000 nM substrate and 10 nM enzyme (100:1, S:E). Error bars denote±standard deviation of the mean for 3 independent replicates (circles). (22B, 22C) Kinetic curve and representative gel of Dz 46 activity under pseudo first-order reaction conditions. The observed rate constant (K_{obs}) is shown. Reported value represents the mean for 2 independent replicates. Reactions were performed in simulated physiological buffer containing 0.25 mM MgCl₂, 50 mM Tris (pH 7.5), 10 mM NaCl, and 140 mM KCl at 37 °C with 0.5 UM substrate and 2.5 μM enzyme (1:5, S:E). Molecular weight markers indicated to the right of the gel. S: 5′-Cy5 labeled full-length substrate, P: 5′-Cy5 labeled cleavage product.

FIGS. 23A-23B are bar graphs showing the nearest neighbor analysis of unmodified Dz 1. (23A) Profiles observed for reactions performed in the presence of 1 mM MgCl₂ and (23B) 5 mM MgCl₂. Normalized initial rates observed for each DNAzyme-substrate pair using 1000 nM substrate and 10 nM enzyme (100:1, S:E) at 37 °C in buffer containing 50 mM Tris (pH 7.5), 10 mM NaCl, 140 mM KCl. Reported values are the mean of 2 independent replicates (circles).

FIGS. 24A-24B show the analysis of Dz 46 cleavage motif preferences in long RNA substrates. (24A) Predicted secondary structures of 60 nt. substrates carrying UGUU (top; SEQ ID NO: 17) or AGUU (bottom; SEQ ID NO: 43) cleavage motifs. Nucleotides complementary to the DNAzyme binding sites are at positions 23-38; cleavage motifs are the 4 positions centered around the arrows; cleavage sites are indicated by arrows. (24B) Representative PAGE gels showing time-dependent cleavage of the substrates shown above. Reactions were performed in simulated physiological buffer containing 1 mM MgCl₂, 50 mM Tris (pH 7.5), 10 mM NaCl, and 140 mM KCl at 37 °C with 500 nM substrate and 50 nM enzyme (10:1, S:E). Molecular weight markers indicated to the right of the gel. S: 5′-Cy5 labeled full-length substrate, P: 5′-Cy5 labeled cleavage product. For each cleavage motif, n=1.

FIG. 25 shows the analysis of Dz 1 cleavage preferences in long RNA substrates. a. Representative PAGE gels showing time-dependent cleavage of the substrates shown in FIG. 24A. Reactions were performed in simulated physiological buffer containing 5 mM MgCl₂, 50 mM Tris (pH 7.5), 10 mM NaCl, and 140 mM KCl at 37 °C with 500 nM substrate and 50 nM enzyme (10:1, S:E). Molecular weight markers indicated to the right of the gel. S: 5′-Cy5 labeled full-length substrate, P: 5′-Cy5 labeled cleavage product. For each cleavage motif, n=1.

FIG. 26 shows the evaluation of allele-specific cleavage by Dz 46 under multiple turnover conditions in the presence of RNase H. Denaturing PAGE gels showing time-dependent RNA cleavage profiles in reactions containing both the wild-type and G12V (aligned with 30 nt marker) substrates (1:1) in the presence of 5 ng/L human RNase H1. Reaction was performed in a buffer containing 1 mM MgCl₂, 50 mM Tris (pH 7.5), 10 mM NaCl, and 140 mM KCl at 37 °C with 500 nM total substrate and 50 nM enzyme (n=1). Molecular weight markers indicated to the right of the gel. S: 5′-Cy5 labeled full-length substrate, P: 5′-Cy5 labeled cleavage product.

FIGS. 27A-27B show the absolute values observed for allele-specific knock-down. (27A) Absolute signals of G12V and WT KRAS mRNA levels observed for cells treated with Dz 46 in a time course of 6 h, 12 h and 24 h. Error bars denote±standard deviation from the mean of 4 replicates (2 biological and 2 technical). (27B) Representative of agarose gel images observed following RFLPA analysis shown in FIG. 6C along with a GAPDH loading control from the same cDNA (2 technical replicates). Molecular weight markers indicated to the right of the gel.

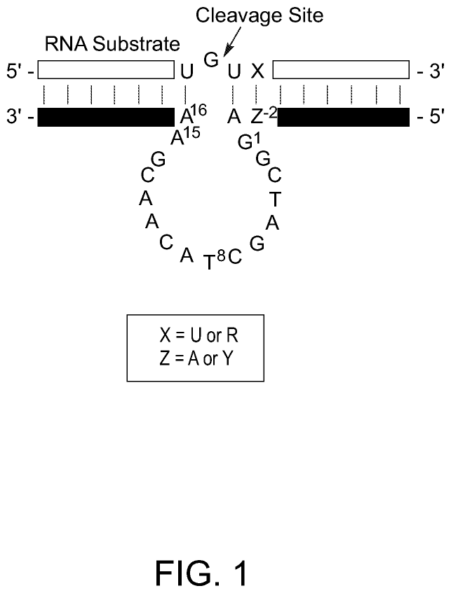

### DETAILED DESCRIPTION

Provided are new molecules, materials, and methods for gene silencing. The modified 10-23 DNAzyme described herein functions with unparalleled catalytic activity under physiological conditions. For the last 25-years, pharmaceutical companies interested in developing 10-23 DNAzymes have determined the best reagents by designing, constructing, and testing 10-23 DNAzymes against all of the G-U dinucleotides in a given mRNA target, a process that is both expensive and laborious. The approach described herein streamlines the drug discovery process by considering the nucleotide positions adjacent to the G-U cut site. Specifically, UGUD motifs (D is A, G, or U, and G-U cut site is underlined) function with the highest catalytic activity, meaning that optimal 10-23 DNAzymes will have an A at position 16 and an A or Y at position-2, where Y is the universal symbol for a pyrimidine (C or T).

### DEFINITIONS

All scientific and technical terms used in this application have meanings commonly used in the art unless otherwise specified. As used in this application, the following words or phrases have the meanings specified.

As used herein, “DNAzyme 10-23” refers to an enzyme comprising a 15-nucleotide (nt) catalytic domain (5′-GGCTAGCTACAACGA-3′ (SEQ ID NO: 1)) that is flanked on both sides by substrate binding arms (i.e., substrate recognition domains) that can vary in length depending on the sequence of the RNA substrate, typically 6-20 nts. In some embodiments, the two substrate recognition domains are designed to achieve target specificity. Note that numbering, when referencing a DNAzyme sequence, is based on the catalytic loop, per convention. For example, position-1 indicates the first position preceding the catalytic loop when viewed in the 5′ to 3′ direction.

As used herein, a “control” or “reference” sample means a sample that is representative of normal measures of the respective marker, such as would be obtained from normal, healthy control subjects, or a baseline amount of marker to be used for comparison. Typically, a baseline will be a measurement taken from the same subject or patient. The sample can be an actual sample used for testing, or a reference level or range, based on known normal measurements of the corresponding marker.

As used herein, a “significant difference” means a difference that can be detected in a manner that is considered reliable by one skilled in the art, such as a statistically significant difference, or a difference that is of sufficient magnitude that, under the circumstances, can be detected with a reasonable level of reliability. In one example, an increase or decrease of 10% relative to a reference sample is a significant difference. In other examples, an increase or decrease of 20%, 30%, 40%, or 50% relative to the reference sample is considered a significant difference. In yet another example, an increase of two-fold relative to a reference sample is considered significant.

“Nucleotide sequence” refers to a heteropolymer of deoxyribonucleotides, ribonucleotides, or peptide-nucleic acid sequences that may be assembled from smaller fragments, isolated from larger fragments, or chemically synthesized de novo or partially synthesized by combining shorter oligonucleotide linkers, or from a series of oligonucleotides.

As used herein, “hybridizes,” “hybridizing,” and “hybridization” means that the oligonucleotide forms a noncovalent interaction with the target RNA molecule under standard conditions. Standard hybridizing conditions are those conditions that allow an oligonucleotide probe or primer to hybridize to a target RNA molecule. Such conditions are readily determined for an oligonucleotide probe or primer and the target RNA molecule using techniques well known to those skilled in the art. The nucleotide sequence of a target polynucleotide is generally a sequence complementary to the oligonucleotide primer or probe. The hybridizing oligonucleotide may contain nonhybridizing nucleotides that do not interfere with forming the noncovalent interaction. The nonhybridizing nucleotides of an oligonucleotide primer or probe may be located at an end of the hybridizing oligonucleotide or within the hybridizing oligonucleotide. Thus, an oligonucleotide probe or primer does not have to be complementary to all the nucleotides of the target sequence as long as there is hybridization under standard hybridization conditions.

The term “complement” and “complementary” as used herein, refers to the ability of two nucleic acid molecules to base pair with each other. In some embodiments, complementarity refers to a nucleotide construct that is capable of base pairing with its target nucleic acid. For example, if a nucleobase at a certain position of a nucleotide construct is capable of hydrogen bonding with a nucleobase at a certain position of a target nucleic acid, then the position of hydrogen bonding between the nucleotide construct and the target nucleic acid is considered to be complementary at that nucleobase pair. Nucleobases comprising certain modifications may maintain the ability to pair with a counterpart nucleobase and thus, are still capable of nucleobase complementarity.

As used herein, “pharmaceutically acceptable carrier” or “excipient” includes any material which, when combined with an active ingredient, allows the ingredient to retain biological activity and is non-reactive with the subject's immune system For gene silencing, representative delivery agents are lipid nanoparticles (LNPs) and ligands for receptor-mediated endocytosis (e.g., GalNac3).

Compositions comprising such carriers are formulated by well-known conventional methods (see, for example, Remington's Pharmaceutical Sciences, 18th edition, A. Gennaro, ed., Mack Publishing Co., Easton, PA, 1990).

As used herein, “treat” or “treatment” or “treating” refers to both therapeutic treatment and prophylactic or preventative measures, wherein the object is to prevent or slow the development of the disease, such as slow down the development of a disorder, or reducing at least one adverse effect or symptom of a condition, disease or disorder. Treatment is generally “effective” if one or more symptoms or clinical markers are reduced, or the progression of a disease is reduced or halted. Thus, “treatment” includes not just the improvement of symptoms or decrease of markers of the disease, but also a cessation or slowing of progress or worsening of a symptom that would be expected in absence of treatment. Beneficial or desired clinical results include, but are not limited to, alleviation of one or more symptom(s), diminishment of extent of disease, stabilized (e.g., not worsening) state of disease, delay or slowing of disease progression, amelioration or palliation of the disease state, and remission (whether partial or total). “Treatment” can also mean prolonging survival as compared to expected survival if not receiving treatment.

As used herein, the term “subject” includes any human or non-human animal. The term “non-human animal” includes all vertebrates, e.g., mammals and non-mammals, such as non-human primates, horses, sheep, dogs, cows, pigs, chickens, and other veterinary subjects. In a typical embodiment, the subject is a human.

As used herein, “a” or “an” means at least one, unless clearly indicated otherwise.

### DNAzymes

The DNAzyme (Dz) described herein has been engineered to target the preferred sequence motif required for optimal cleavage of an RNA sequence. As described herein, structural constraints imposed by chemical modifications drive catalysis toward a highly preferred UGUU/R motif (cut site underlined) that has been validated across several mRNA targets. This DNAzyme provides a tool for precision allele-specific gene silencing, as well as diagnostic and therapeutic applications.

Described herein is a nucleotide construct comprising: a 3′ substrate binding arm; a 5′ substrate binding arm and a catalytic domain. The catalytic domain has the nucleic acid sequence of positions 1-15 of SEQ ID NO: 1, and is disposed between the 3′ substrate binding arm and the 5′ substrate binding arm. The 3′ substrate binding arm and the 5′ substrate binding arm are complementary to nucleotides flanking a G-U cleavage site in a target mRNA, wherein the first nucleotide 3′ of the catalytic domain is an adenosine (A), and wherein the second nucleotide 5′ of the catalytic domain is an A or a pyrimidine (Y). The sequence of the nucleotide construct is thus targeted to a G-U cleavage site, which cleavage site is a U-G-U-U or a U-G-U-R sequence. R, as is understood in this context, refers to a purine nucleotide. A G-U cleavage site in the context of such a 5′-UGUU/R-3′ motif is up to 5-fold more active than 5′-XGUU-3′ motifs, where X corresponds to a G, A, or C residue.

In some embodiments, the substrate binding arms comprise one or more modified nucleic acids. Such modified nucleic acids can be at any position within the substrate binding arms. In some embodiments, a first modified nucleic acid is disposed at the terminus of the 3′ substrate binding arm, a second modified nucleic acid is disposed at the terminus of the 5′ substrate binding arm, and three OMe modifications are positioned adjacent to each of the modified nucleic acids.

In some embodiments, the modified nucleic acids comprise a MOE, locked nucleic acid (LNA), threose nucleic acid (TNA), mannitol nucleic acid (MNA), serinol nucleic acid (SNA), glycol nucleic acid (GNA), hexitol nucleic acid (HNA), diaminopurine (DAP), C-5 modified pyrimidine, pseudo uridicine, or C-5-methyl deoxycytidine. In some embodiments, the modified nucleic acid is an LNA or MOE. MOE is an equivalent, if not slightly better, modification at the termini. MOE is expected to be less toxic than LNA.

In some embodiments, the 3′ substrate binding arm and the 5′ substrate binding arm are each at least 5 nucleotides in length. In some embodiments, the 3′ substrate binding arm and the 5′ substrate binding arm are each 6 to 10 nucleotides in length. In some embodiments, the substrate binding arms are each about 8 nucleotides in length. In some embodiments, the 3′ substrate binding arm and the 5′ substrate binding arm are complementary to nucleotides flanking a G-U cleavage site in a target RNA.

In some embodiments, the target RNA is Kirsten Rat Sarcoma Viral Oncogene Homolog (KRAS) mRNA. Other examples of a target RNA include, but are not limited to, viral or bacterial sequences, such as sequences specific to SARS-COV-2 or other pathogens, including particular strains or subtypes of interest.

### Methods

The Dz nucleotide construct described herein can be used in a method of cleaving a target RNA sequence. In some embodiments, the method comprises contacting the target RNA sequence with a nucleotide construct as described herein. Representative examples of target RNA include, but are not limited to, mRNA and viral RNA. In some embodiments, the 3′ substrate binding arm and the 5′ substrate binding arm are complementary to nucleotides flanking a G-U cleavage site in KRAS mRNA. Other examples of a target RNA include, but are not limited to, viral or bacterial sequences, such as sequences specific to SARS-COV-2 or other pathogens, including particular strains or subtypes of interest, as well as common targets for gene silencing, such as hTTR, PCSK9, SOD1, TAU, SNCA, LRRK2, HTT, SMN2, GATA3, c-JUN, Bcl-2, VEGF, AKT1, and others known to those skilled in the art, such as targets associated with genetic diseases, cardiometabolic diseases, infectious diseases, central nervous system (CNS) and ocular diseases. Representative examples include tau protein as a target associated with Alzheimer's disease, huntingtin as a target associated with Huntington's disease, and SOD1 as a target associated with Amyotrophic Lateral Sclerosis (ALS), among others.

Additionally described is a method of modulating the translation of a disease-associated protein. Modulating, or disrupting, the translation of the disease-associated protein has the effect of silencing the corresponding gene. In some embodiments, the method comprises contacting a target mRNA sequence that encodes the disease-associated protein with a nucleotide construct as described herein. Examples of disease-associated proteins include, but are not limited to, cancer-related proteins.

Also provided is a method of treating a disease or condition or ameliorating a symptom thereof. In some embodiments, the method comprises administering an effective amount of a nucleotide construct as described herein to a subject in need thereof. In some embodiments, the disease or condition is caused by a genetic disease, viral or bacterial pathogen, cancer, inflammation, cardiovascular disease, immune deficiency, or a neurological disorder. In some embodiments, the cancer is associated with KRAS mutations. In some embodiments, the disease or condition is lung, pancreatic, or colorectal adenocarcinoma, or melanoma.

Further provided is a method of detecting the presence of a target RNA sequence in a sample. In some embodiments, the method comprises contacting the sample with a nucleotide construct as described herein, wherein the 3′ substrate binding arm and the 5′ substrate binding arm of the nucleotide construct are complementary to the target RNA sequence. The method further comprises measuring cleavage of the target RNA sequence, wherein cleavage is indicative of presence of the target RNA sequence in the sample. In some embodiments, the nucleotide construct is modified with a marker that is detectable upon cleavage, and wherein the measuring comprises measuring the presence of the detectable marker. In some embodiments, the detectable marker is a fluorophore. For example, a quencher may be positioned in the construct such that, upon cleavage, the fluorophore is no longer subjected to quenching, thereby resulting in detectable signal. A representative example of a detection method that employs such a DNAzyme is described in Yang et al., 2022, Journal of the American Chemical Society 2022 144 (26), 11685-11692 DOI: 10.1021/jacs.2c03420. The SARS-COV-2 region of interest can be isothermally amplified by RT-RPA and T7 RNA polymerase to produce multiple copies of the RNA analyte, or optionally, detection can be achieved without pre-amplification. Competitive DNAzyme assembly on the viral RNA produces a fluorescent signal specific to the sample genotype via site-specific cleavage of a quenched fluorescent reporter.

### EXAMPLES

The following examples are presented to illustrate the present invention and to assist one of ordinary skill in making and using the same. The examples are not intended in any way to otherwise limit the scope of the invention.

### Example 1: Chemical Evolution of an Autonomous DNAzyme with Allele-Specific Gene Silencing Activity

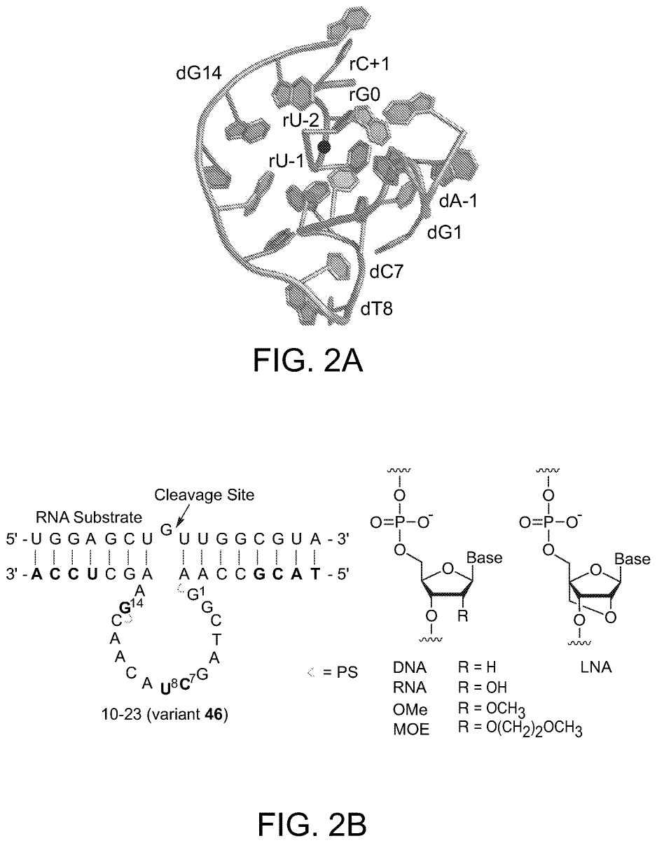

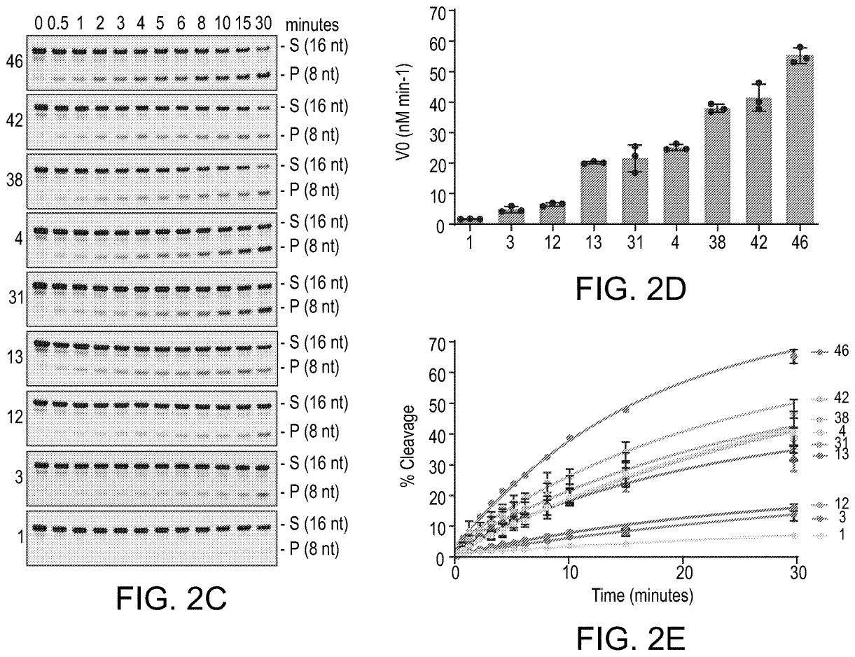

This Example describes the chemical evolution of a highly modified 10-23 DNAzyme that functions with strong catalytic activity under near-physiological conditions. The enzyme was discovered through iterative cycles of design that were guided by structural information available on the folding topology and metal-ion binding sites of Dz 10-23 (22). The enzyme, termed Dz 46 (FIG. 2B), can achieve ~65 turnovers in 30 min, making it a highly effective RNA-cleaving DNAzyme under near-physiological conditions. Structural constraints imposed by the chemical modifications drive catalysis toward a highly preferred UGUD motif (cut site underlined, and D is the IUBMB nucleotide code for A, G, or U) that was validated across several mRNA targets (23). Detailed biochemical assays, including quantification of cellular RNase H1 levels, support an autonomous RNA cleavage mechanism with minimal RNase H1 engagement. Consistent with its strong catalytic activity, the enzyme exhibits persistent allele-specific gene silencing activity in cultured mammalian cells, endogenously expressing a currently undruggable G12V mutant KRAS target. These results demonstrate that chemical evolution offers a powerful approach to discovering new chemotype combinations that can be used to engineer DNAzymes with physicochemical properties that will help drive clinical applications.

### Materials

Oligonucleotide sequences used in this study are listed in Supplementary Tables 1~4 of Nguyen et al., Nature Communications (2023) 14:2413. All oligonucleotides were purchased from Integrated DNA Technologies (Coralville, Iowa), except for Dz 6 and Dz 7, which were prepared by solid-phase oligonucleotide synthesis using an ABI 3400 automated DNA synthesizer. The TNA-G phosphoramidite used for Dz 7 was chemically synthesized as described previously (45). All other reagents used for solid-phase synthesis were purchased from Glen Research. In-house oligonucleotides were synthesized in DMT-on mode, desalted, and purified by reverse-phase HPLC. Oligonucleotides purchased from IDT were used directly without further purification. Stock solutions used to prepare reaction buffers were purchased as follows: 1 M Tris-HCl PH 7.5 (Quality Biological), 1 M MgCl₂ (Invitrogen), 2 M KCl (Invitrogen), and 5 M NaCl (Sigma). Molecular and cellular biology reagents include Dulbecco's modified eagle medium, DMEM (ThermoFisher Scientific), RPMI 1640 medium, and NCI-H441 lung adenocarcinoma (ATCC, Cat #: ATCC-CRM-HTB-174™), jetPRIME® DNA and siRNA transfection reagent (Polyplus Transfection), Trizol reagent (Invitrogen). RNA clean & concentration-5 kit (Zymo Research), Turbo DNase and SuperScript III First-strand Synthesis System (Invitrogen-Life Technologies, CA), E. coli RNAse HI and BstNI (NEB), Human RNAse H1 (Abcam, Cat #Ab153634). Bichinconinic Acid (BCA) assay (GBioSciences), Human Ribonuclease H ELISA kit (AssayGenie, Cat #HUDL02549) In vitro cleavage reaction

Totally, 10-23 variants were assayed under multiple turnover conditions using either 10:1 (S:E, 500 nM RNA substrate and 50 nM DNAzyme) or 100:1 (S:E, 1000 nM RNA substrate and 10 nM DNAzyme) in simulated physiological reaction buffer containing 50 mM Tris-HCl (pH 7.5), 10 mM NaCl, 140 mM KCl, and 1 mM MgCl₂, unless otherwise noted. DNAzymes and substrates were annealed in the absence of MgCl₂ by heating for 5 min at 95 °C and cooling for 5 min at 4 °C. Solutions were equilibrated to 37 °C for 2 min before initiating the reaction with the addition of MgCl₂ and kept at 37 °C throughout the time course. Aliquots (1.5 μL) of the reaction mixture were removed at the indicated time points and quenched in 16.5 μL stop buffer (99% deionized formamide, 25 mM EDTA). Quenched samples were denatured at 95 °C for 10 min and analyzed by 20% denaturing PAGE. Gels were imaged using an Odyssey CLx imaging system (LI-COR) and quantified using Image Studio Lite (LI-COR).

### Initial Velocity (v₀) Determination

Initial velocity (v₀) was measured between 0 and 30 min under 100:1 (S:E) multiple turnover conditions as described above. Values for v0 were calculated from a linear fit of the first 10-15% of the cleavage reaction using Eq. (1)

$${v}_{0} = \frac{( {Y}_{f} - {Y}_{0} ) \times [ {S}_{0} ]}{( {t}_{f} - {t}_{0} )} \qquad ( 1 )$$

where v0 is the initial velocity (nM*min⁻¹), Y_{f} is the percent cleavage at finite time t when the first 10-15% cleavage is reached, Y₀ is the percent cleavage at t₀, and [S₀] is the initial substrate concentration (nM) at t₀.
   Rate Constant (k_{obs}) Determination

Single turnover experiments were performed at 37 °C under pseudo-first order reaction conditions using a 1:5 ratio of S:E (0.5 μM substrate and 2.5 UM enzyme) in a buffer containing 50 mM Tris-HCl (pH 7.5), 10 mM NaCl, 140 mM KCl, and 0.25 mM MgCl₂. Aliquots of the reaction mixture were collected and resolved by PAGE, as described previously. Observed rate constants were obtained by fitting the percentage of the cleaved substrate over the reaction time (min) to the one-phase association Eq. (2) using Prism 9 (GraphPad, USA)

$$Y = {Y}_{0} + ( {Y}_{∞} - {Y}_{0} ) \times ( 1 - {e}^{- {k}_{obs} \times t} ) \qquad ( 2 )$$

where Y is the percent cleavage at finite time t, Y₀ is the percent cleavage at t₀, Y∞ is the percent cleavage at an infinite time where the reaction plateaus and K_{obs} is the observed pseudo-first-order rate constant (min⁻¹).

### Data Analysis Software

Prism 9 (Graphpad, USA) for curve fitting and Microsoft Excel was used to generate plots.

### Mg²⁺ Dependency

Mg²⁺ dependency was measured under multiple turnover conditions as described above using 100:1 (S:E) in cleavage reactions containing defined concentrations of MgCl₂ (0, 0.10, 0.25, 0.50, 1.0, 2.5, and 5.0 mM). After 20 min of reaction, aliquots (1.5 μL) were removed, quenched, and analyzed by 20% denaturing PAGE as described in the in vitro cleavage reaction section. Similar assays were performed in the presence of 1 mM ATP to assess the cleavage activity of Dz 46 in the presence of a natural Mg²⁺ chelator.

### Substrate Specificity Analysis

To determine the preferred substrate cleavage motif, a nearest neighbor analysis was performed by measuring the initial rates for matched Dz-substrate pairs containing single nucleotide changes to the 5′ and 3′ positions adjacent to the GU cleavage site in the RNA substrate. Reactions were performed as described in the initial rate section above. For each Dz-substrate pair, the initial velocity was calculated and normalized to the fastest motif.

### RNase H1 Evaluation

The contribution of RNase H toward substrate cleavage was evaluated using E. coli RNase HI and human RNase H1. For the E. coli RNase HI assay, the long (60 nt) G12V KRAS RNA substrate (250 nM) was incubated at 37 °C for 10 min in the presence of 50 mM Tris-HCl (pH 7.5), 10 mM NaCl, 140 mM KCl. Reactions were initiated with a final concentration of 1 mM MgCl₂, 0.05 U/μL E. coli RNase HI or water for RNase H free controls, and 250 nM of either Dz 46 (active), Dz 47 (inactive), or a linear 22 nt all-DNA ASO. Aliquots (1.5 μL) were removed at 30 min, quenched, and analyzed by 8% denaturing PAGE as described above. The human RNase H1 assay was performed similarly to the E. coli assay, with the exception that human RNase H1 levels spanned the biological range of 0-5 ng/μL, as determined by ELISA (see below).

### Allele-Specific RNA Cleavage In Vitro

Cell-free allele-specific cleavage assays were performed using the long (60 nt) versions of the wild-type and G12V KRAS RNA substrate (250 nM) in two formats: alone or in competition with one another.

Individual format. DNAzyme targeting specificity was assessed using multiple turnover conditions (10S: 1E): 500 nM of the long (60 nt) versions of either WT or G12V KRAS RNA substrate was mixed with 50 nM Dz 49 (WT, active) or Dz 46 (G12V, active) in the buffer containing 50 mM Tris-HCl (pH 7.5), 10 mM NaCl, 140 mM KCl, 1 mM MgCl₂ and incubated at 37 °C. Aliquots of 1.5 μL reaction mixture were taken at 5 min and 30 min, quenched, and resolved using 8% denaturing PAGE as described in the in vitro cleavage reaction section.

Competition format. Cleavage activity was assessed using 250 nM of Dz 46 (active), Dz 47 (inactive), Dz 50 (active, non-binding), or ASO (48) and mixed with two 60 nt RNA substrates in a 1:1 ratio: 125 nM WT KRAS with a 5′Cy5 tag and 125 nM G12V KRAS with a 5′Alexa Fluor 750 tag. Reactions were performed at 37 °C in buffer containing 50 mM Tris-HCl (pH 7.5), 10 mM NaCl, 140 mM KCl, and 1 mM MgCl₂, with or without 5 ng/μL human RNase H1. Aliquots (1.5 μL) were removed after 30 min, quenched, and resolved using 20% denaturing PAGE as described in the previous section.

### Intracellular Assays

Cell lines and mammalian cell culture conditions. NCI-H441 (Cat. #CRM-HTB-174), HEK293 (Cat. #CRL-1573), and K562 (Cat. #CCL-243) cell lines used in this study were purchased from American Type Culture Collection (ATCC). HEK293 cells were cultured in DMEM. K562 and NCI-H441 cells were cultured in RPMI 1640. Media were supplemented with 10% fetal bovine serum and 1% penicillin-streptomycin (1 mg/mL) and grown at 37 °C, 5% CO₂.

Enzyme-linked immunosorbent assay (ELISA). HEK293 and K562 cells (3 aliquots/cell line) and NCI-H441 cells (2 aliquots) were harvested, counted, and re-suspended in 1×PBS pH 7.4 supplemented with DNase, RNase A, and proteinase inhibitor cocktail set VII and subjected to protein extraction and BCA for quantification. HsRNase H1 concentration from the total protein of the three cell lines was determined using the human ribonuclease H ELISA kit according to the manufacturer's instruction and quantified using a Clariostar Plus microplate reader (BMG Labtech, NC).

Transfection. After 48-96 h post-seeding and when cell density reached 60%, NCI-H441 cells in 6-well plates were transfected with transfection carrier only (0, Negative control), 50 or 500 nM of Dz 46 using JetPrime transfection reagent according to manufacturer's instruction except for a higher amount (5×) of transfection reagent. For negative controls, the volume of JetPrime reagent used for each well was the same as those with DNAzymes to ensure the same transfection condition in the control and experimental samples. At 6, 12, and 24 h post transfection, cells were harvested and subjected to total RNA extraction.

RNA isolation and reverse transcription (RT). To each well of a 6-well plate, total RNA isolation was performed using 1 mL/well Trizol reagent according to the manufacturer's instructions. Total RNA was treated with Turbo DNase (20 U/reaction) at 37 °C for 30 min with shaking, followed by purification using RNA clean and concentration-5 kit according to manufacturer's instructions. Total RNA was eluted twice with 22 μL per elution, resulting in 44 μL total volumes. DNA-free RNA (700 ng) was subjected to cDNA synthesis using SuperScript III First-strand Synthesis System with random hexamer primers in a 20 μL reaction according to the manufacturer's instructions. Template mRNA in the RNA:cDNA hybrid was removed by RNase H (2U/cDNA reaction) digestion resulting in a final volume of 21 μL.

Restriction fragment length polymorphism assay (PCR-RFLPA). PCR-RFLPA was performed as previously described with the following changes (37). In brief, upon cDNA synthesis, amplification of 200 nt KRAS fragment was performed using a unique sense primer to introduce a single substitution (G to C) at the first nucleotide (bold) of KRAS codon 11 (GCT to CCT). This substitution introduces the BstNI recognition site (“CCWGG”) spanning from codon 11 (CCT) to codon 12 (GGT) of the KRAS WT allele only. G12V KRAS allele has a mutation (bold) at codon 12 (GGT to GTT) and thus resists BstNI cleavage. The antisense primer is oKN483R: 5′-TCCTCTTGACCTGCTGTGTCG-3′ (SEQ ID NO: 44), and the sense primer is oKN484F: 5′-TTATTATAAATAATGACTGAATATAAACTTGTGGTAGTTG GAcCT-3′ (SEQ ID NO: 45) with a lower case “c” denotes a substitution. 5 μL of unpurified cDNA was used as a template for PCR (40 cycles), and the amplicon was subjected to purification. 500 ng of the amplicon was digested with 20 U BstNI for 1 h at 60 °C in a 50 μL reaction. The G12V allele remained intact (200 nt). WT allele was cleaved and resulted in two fragments: 150 nt and 50 nt. Upon resolving in a 3% agarose gel (132 ng/lane), WT and G12V alleles were separated by a difference of 50 nt. For loading control, 1 μL of unpurified cDNA was used as a template for GAPDH amplification (PCR: 20 cycles). Amplicons were purified using Zymo DNA-Clean and Concentration-5 kit (Zymo, Cat #D4003) and eluted with nuclease-free water twice (22 μL/each). GAPDH primers are oKN60F: 5′-ACCATCTTCCAGGAGCGAGATCCCTC-3′ (SEQ ID NO: 46) and oKN61R: 5′-TGCAGGAGGCATTGCTGATGATCTTGA-3′ (SEQ ID NO: 47).

### Statistics and Reproducibility

Descriptive statistics were calculated in Microsoft Excel. Experiments were generally reproducible. No statistical method was used to predetermine the sample size.

### Results

### Chemical Evolution of Dz 10-23

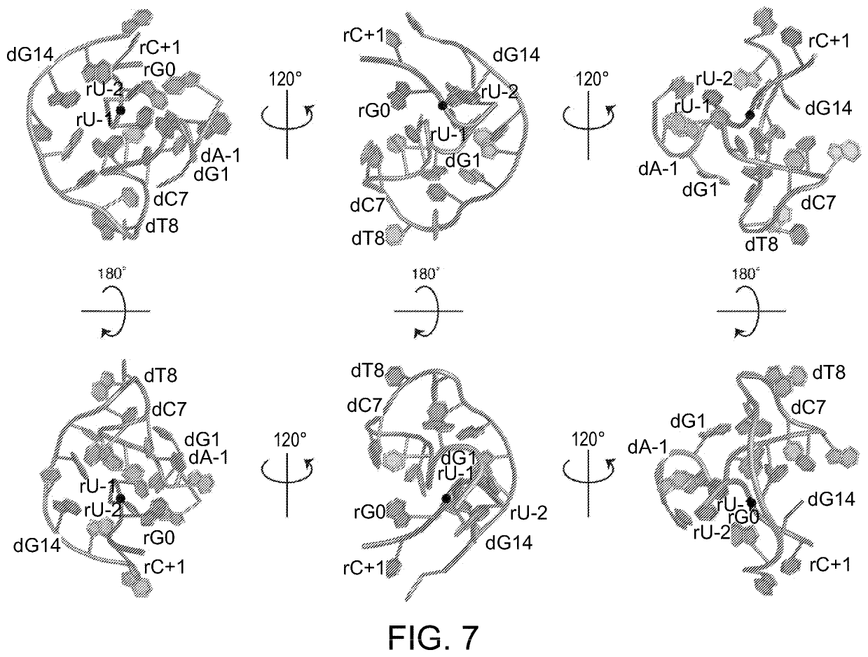

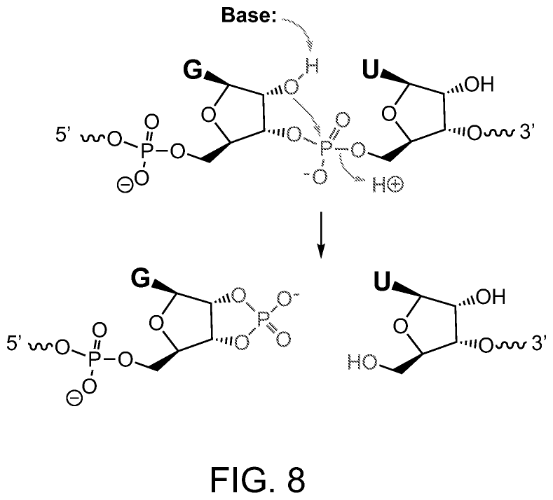

Recent structural insights into the folding and dynamics of Dz 10-23 (FIG. 2A, FIG. 7) provide a valuable starting point for designing next-generation DNAzymes that can function with improved stability and catalytic activity (22). In particular, topological changes caused by metal-ion-induced conformational plasticity illuminate the importance of dG₁₄ as a critical residue in the catalytic loop. Molecular dynamics (MD) simulations indicate that the O2′ atom of the scissile rG⁰ residue on the RNA substrate interacts with the catalytic dG₁₄ residue 5-times more frequently than its direct interactions with a hydrated Mg²⁺ ion present in metal-ion-binding site III (22). This observation suggests that 10-23 functions by an acid-base mechanism, whereby dG₁₄ abstracts a proton from the O2′ atom of the scissile rG⁰ residue, while a hydrated Mg²⁺ ion simultaneously donates a proton to the 05′ atom of the adjacent U⁻¹ residue. An in-line attack of the O2′ oxyanion on the adjacent phosphodiester bond leads to the formation of an upstream cleavage product carrying a cyclic 2′,3′-monophosphate, and a downstream strand with a 5′ hydroxyl group (FIG. 8). Recognizing that the in-line conformation responsible for the proposed acid-base mechanism was observed with low frequency in MD simulations (22), we postulated that synthetic congeners of DNA introduced at position dG14 and elsewhere in the scaffold could lead to improved catalytic activity by shifting the equilibrium of the various 10-23 topologies in favor of a catalytically active conformation.

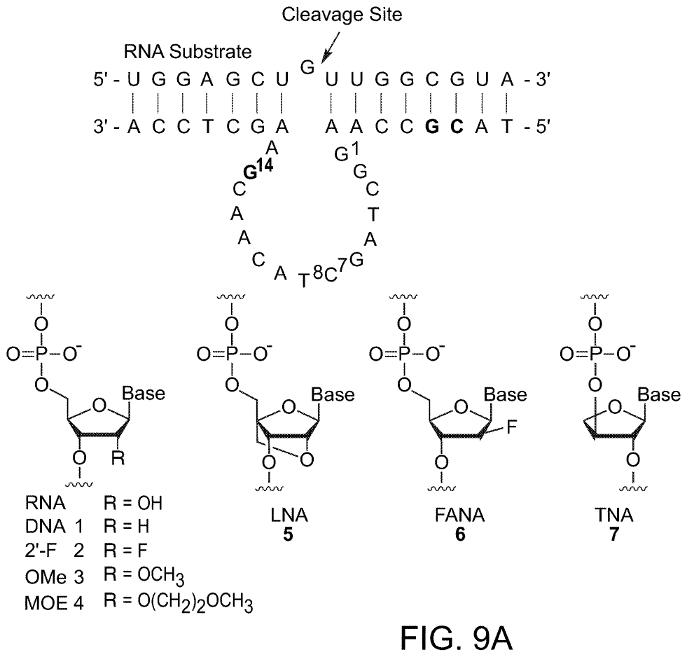

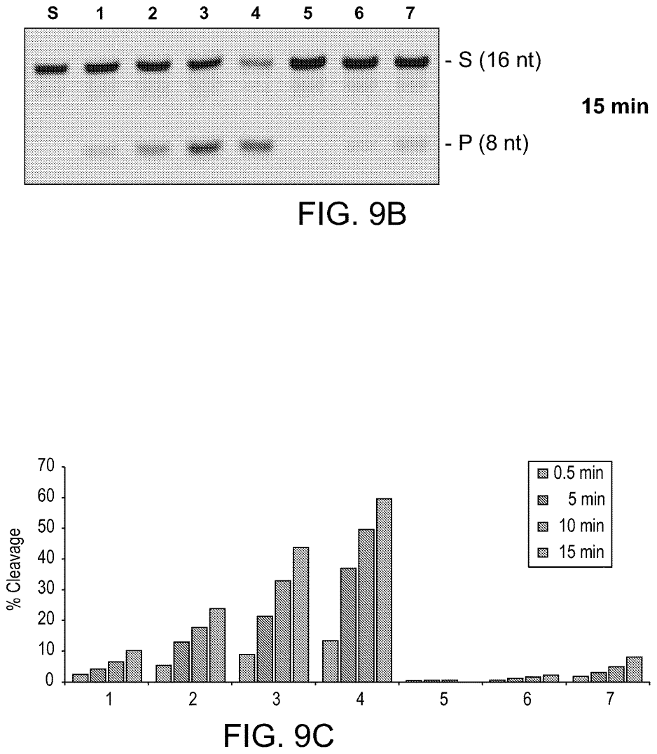

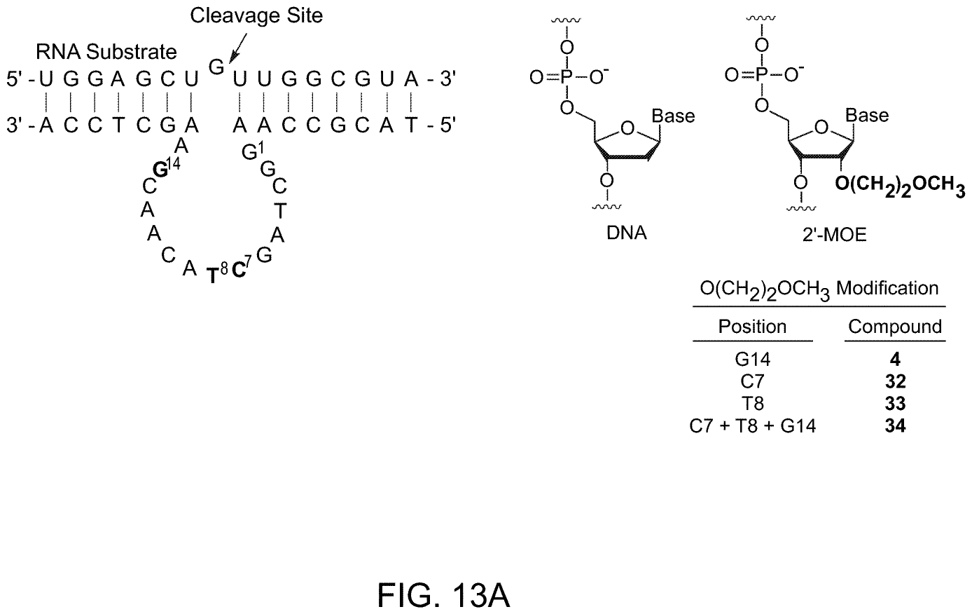

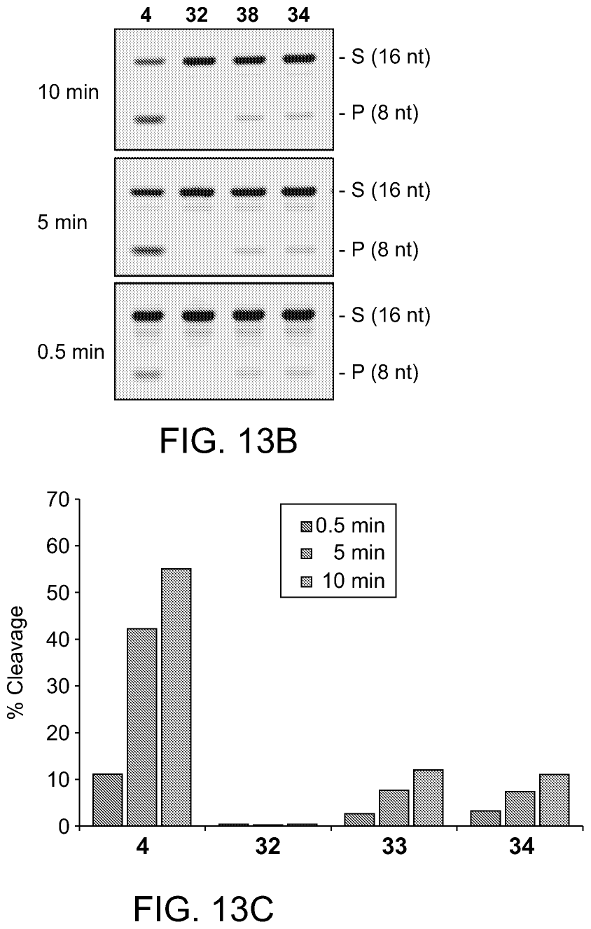

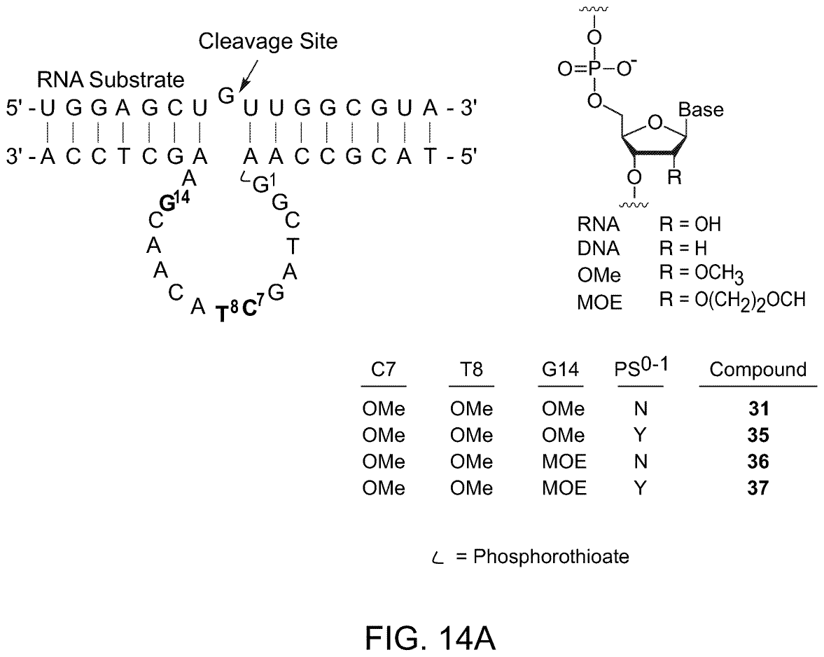

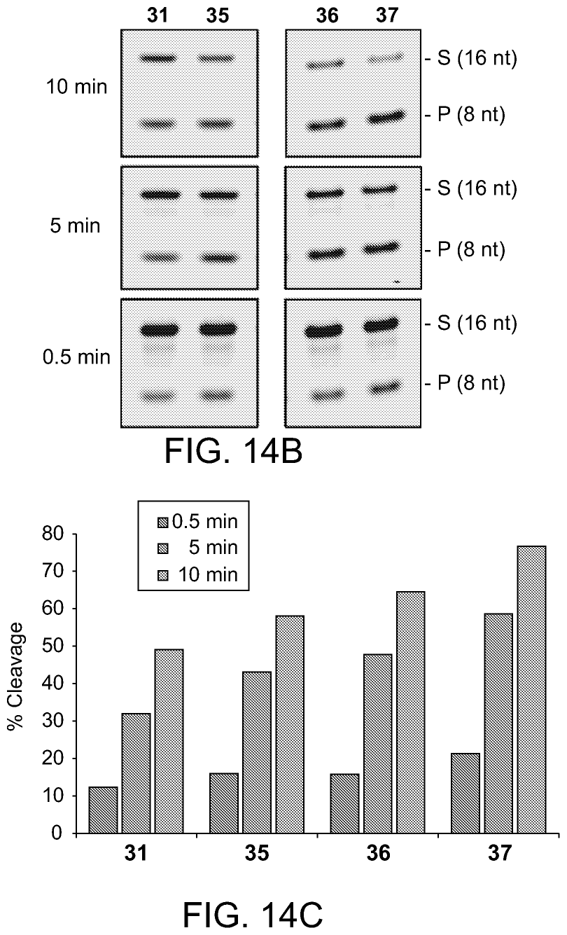

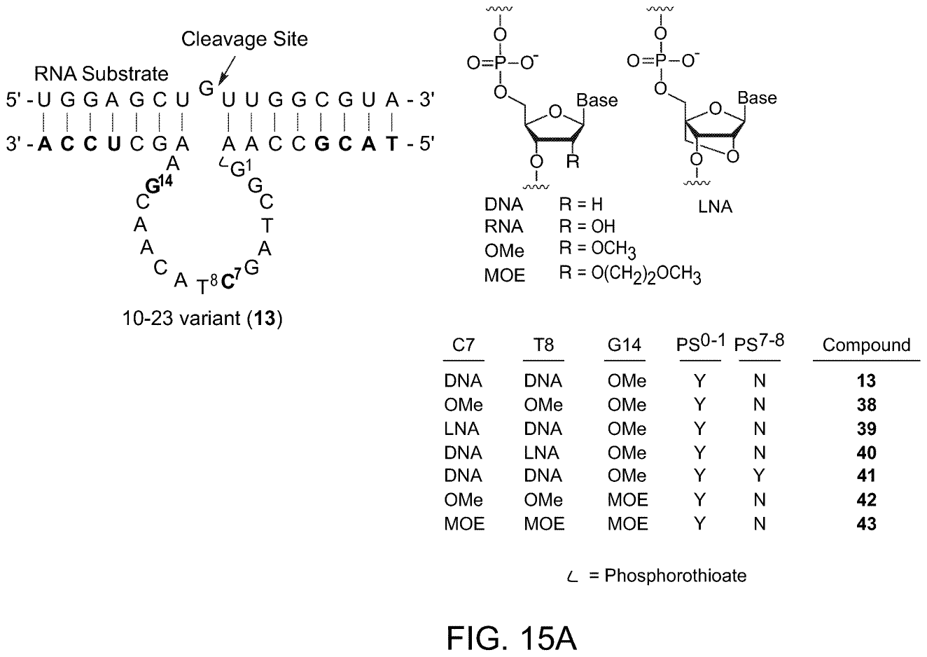

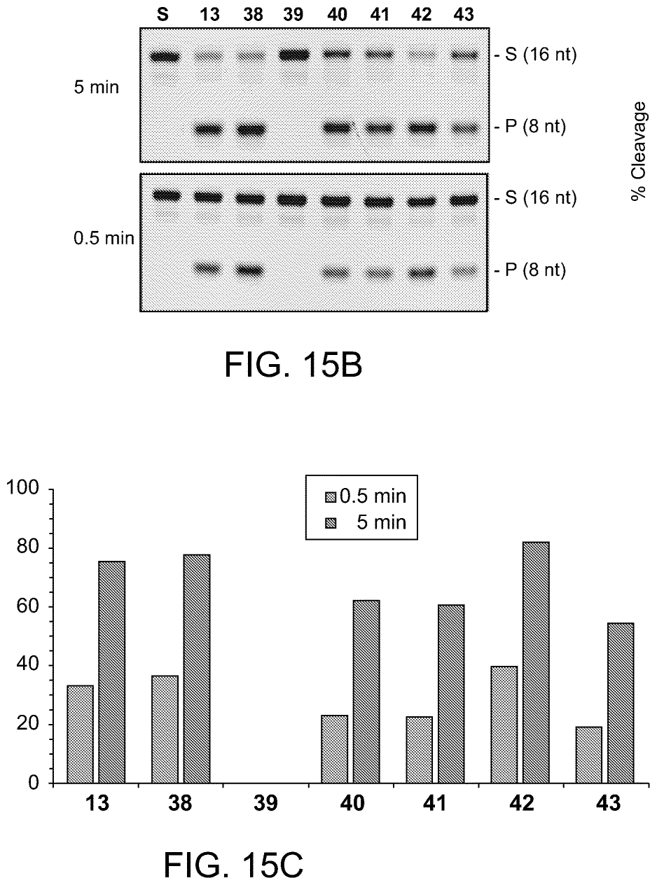

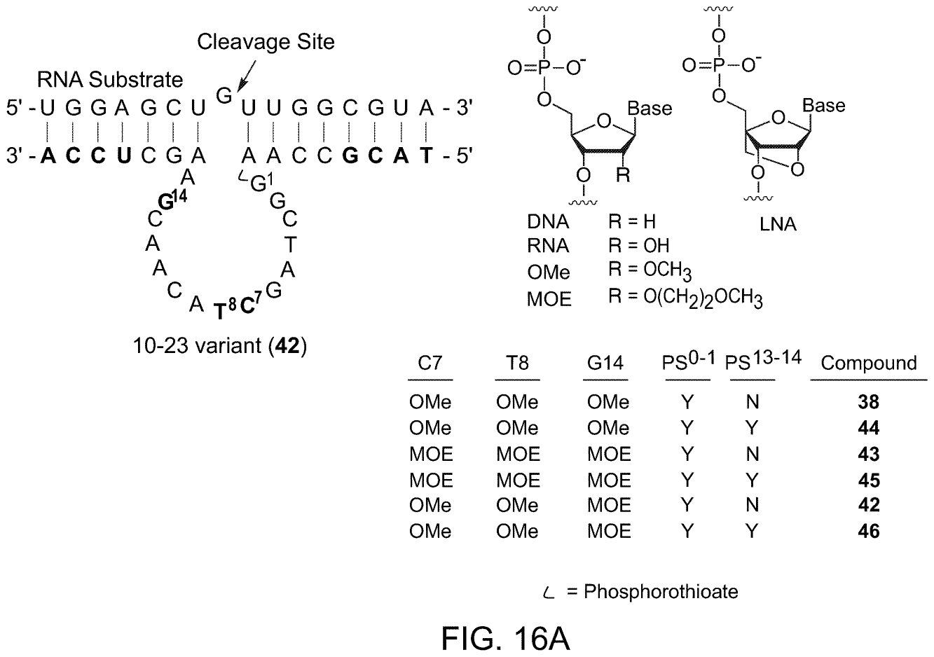

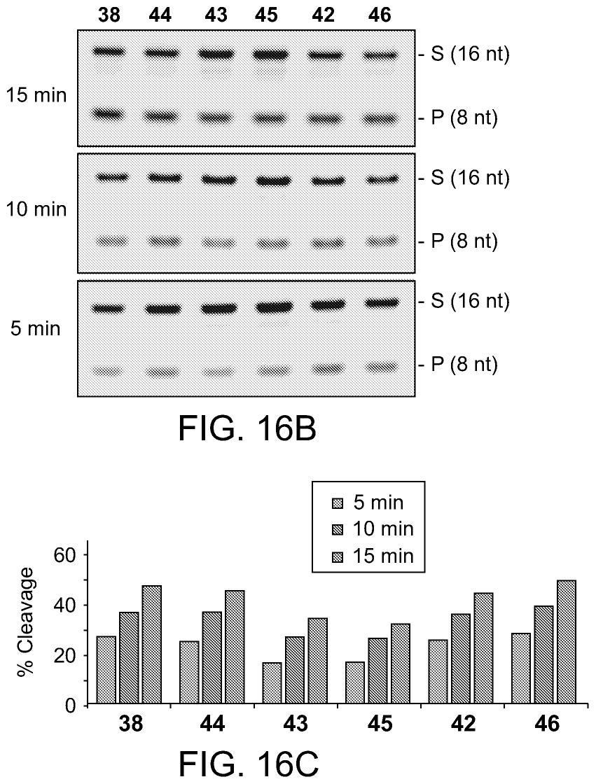

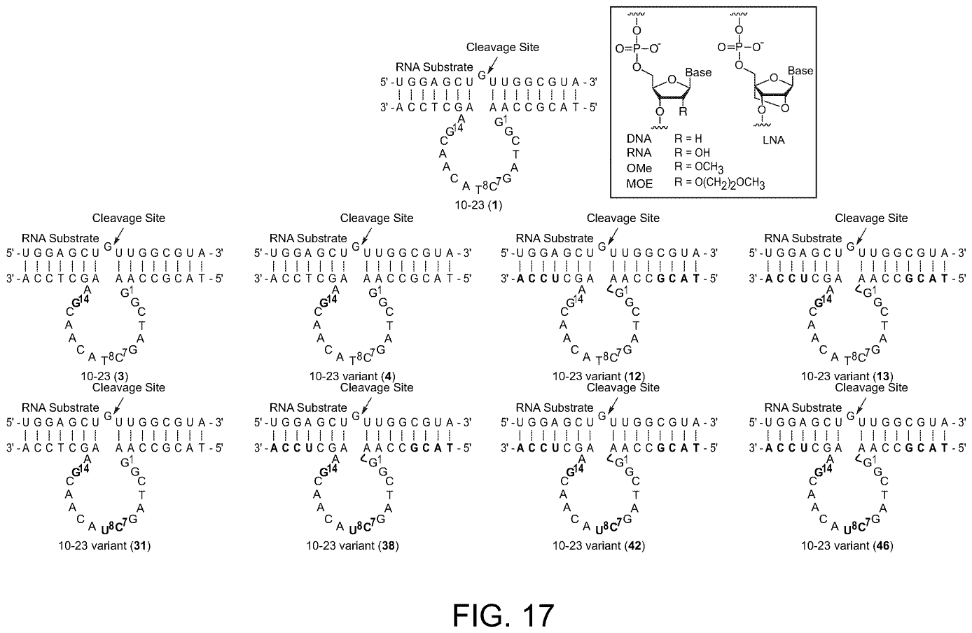

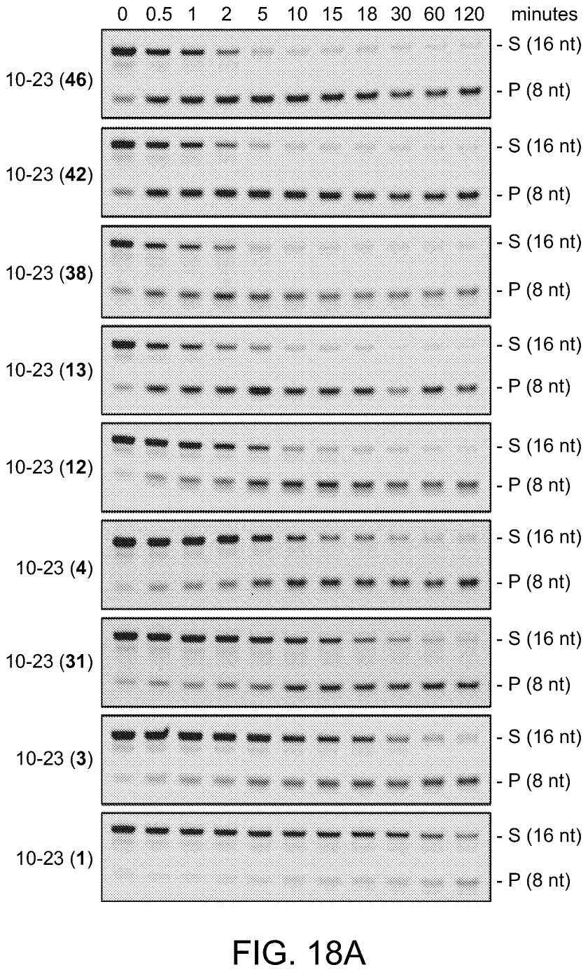

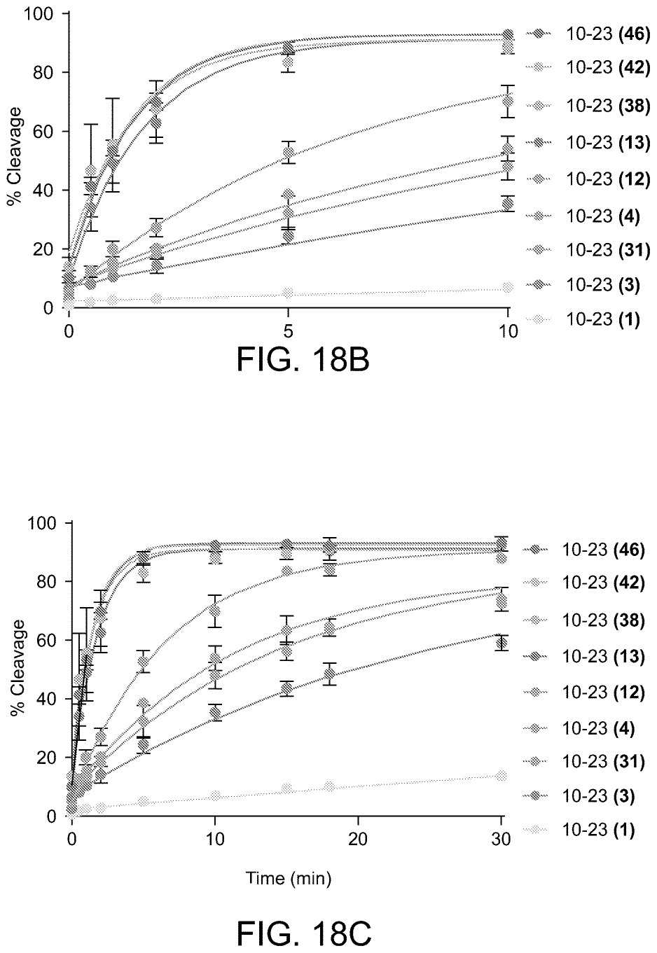

We envisioned a chemical evolution process in which the classic Dz 10-23 framework would be optimized through a series of iterative design, build, test, and learn cycles intended to uncover permissive sites in the scaffold where chemical modifications could contribute favorably to catalytic activity. We focused our designs around an 8+7 binding arm configuration, which provided a balanced solution to the problem of how to enhance enzyme kinetics while avoiding product inhibition caused by increased thermodynamics of substrate binding. We recognized that certain chemical modifications, especially those made to the bind arm region, could alter the equilibrium between the pre- and post-catalytic state and therefore sought to identify synthetic congeners that would maximize multiple turnover activity through optimal RNA binding. To ensure that the best designs functioned in a cellular context, all of the optimization assays were performed under multiple turnover conditions in buffer meant to simulate the cellular environment [1 mM MgCl₂, 140 mM KCl, 10 mM NaCl, and 50 mM Tris-HCl (pH 7.5)] at 37 °C. The functional impact of each chemical perturbation toward Dz 10-23 activity was measured by denaturing polyacrylamide gel electrophoresis (PAGE) (FIGS. 9-16, Tables 1-4). As the chemical complexity of our variants increased, the substrate(S) to enzyme (E) ratio was increased from 10:1 to 100:1 (S:E) so that we could accurately distinguish the activity of our best variants, some of which function on the timescale of seconds under the less stringent conditions of the 10-fold excess substrate (FIGS. 17 and 18).

Influenced by the NMR structure of Dz 10-23, we began by evaluating the RNA cleavage activity of 10-23 variants carrying synthetic congeners at position dG₁₄ of the catalytic loop. Substitution of the 2′-deoxyguanosine nucleotide for a panel of base-matched XNA residues bearing alternative sugar moieties revealed a strong preference (~5-fold) for 2′-O-methoxyethylribonucleic acid (MOE) (FIG. 9). This observation implies that the catalytically active conformation prefers an A-type sugar pucker at position 14, as MOE-modified nucleotides exclusively adopt a 3′-endo sugar conformation due the strong gauche effect imposed by the 2′ substituent. Modest activity gains were also observed for OMe and 2′-fluororibonucleic acid (F-RNA) substitutions, while LNA, FANA, and threose nucleic acid (TNA) substitutions were less active than the native DNA residue, presumably because these residues distort the geometry required for an in-line attack mechanism.

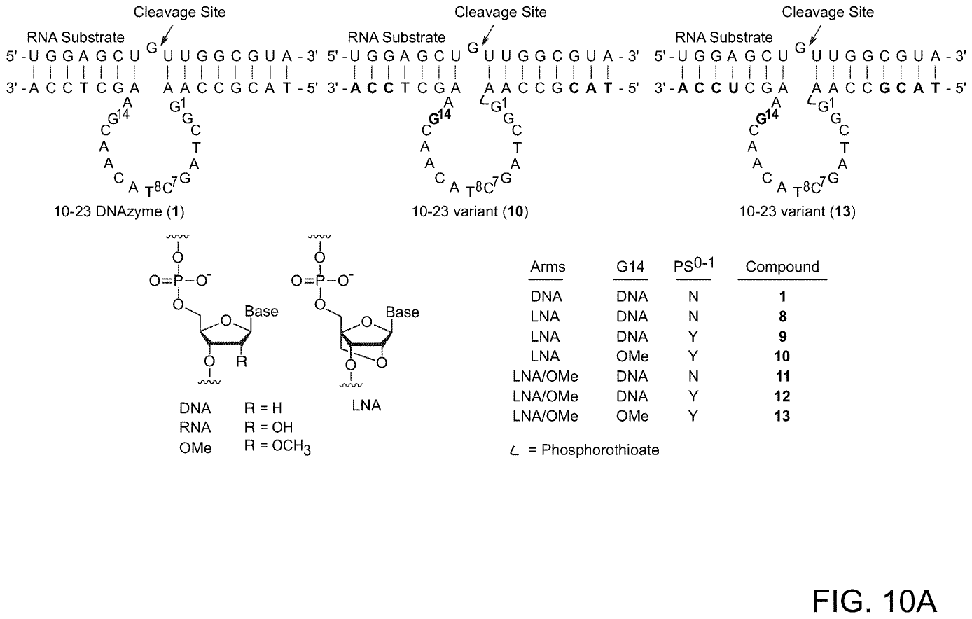

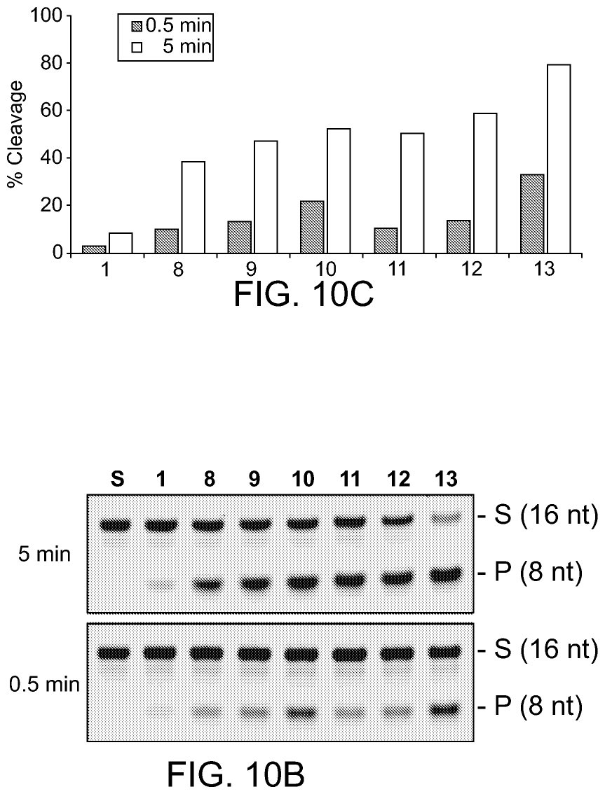

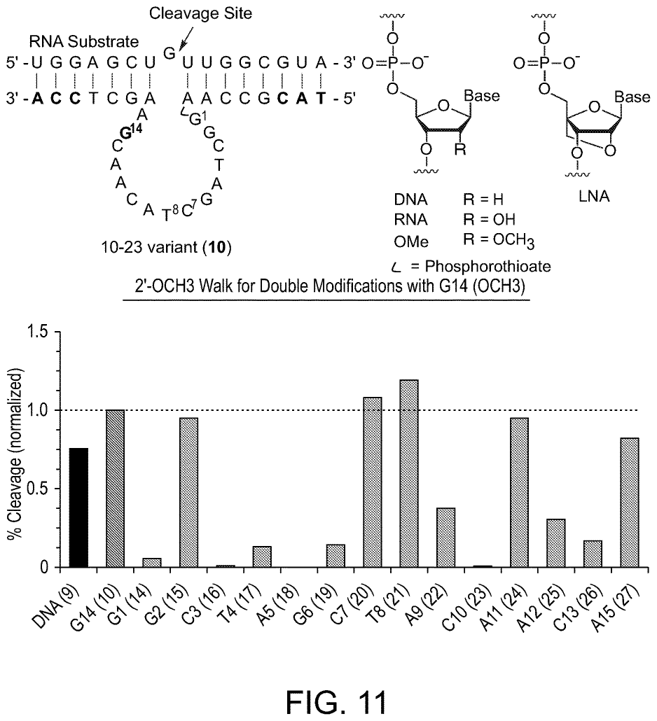

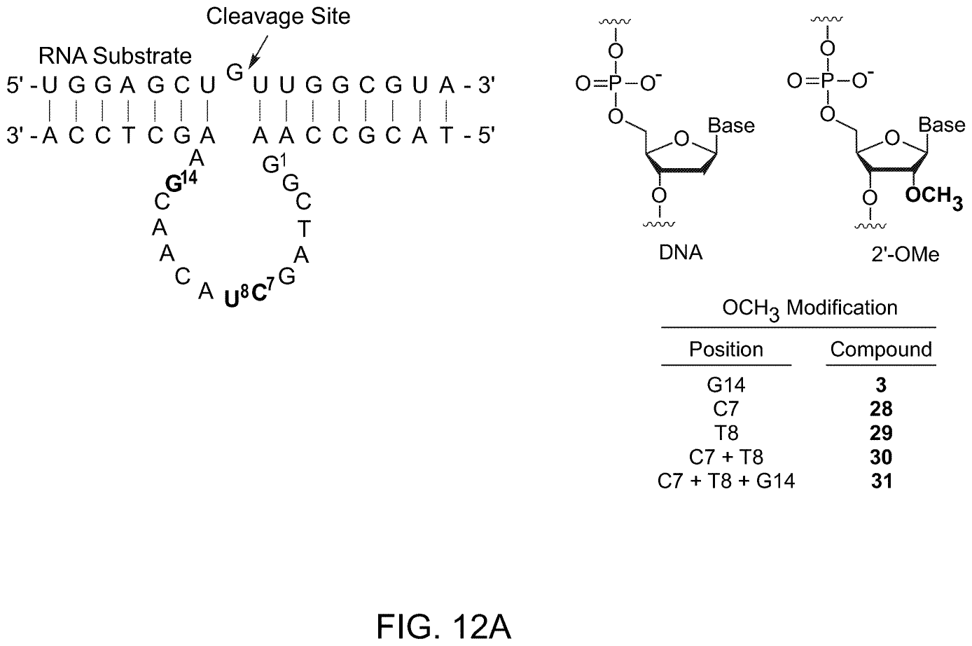

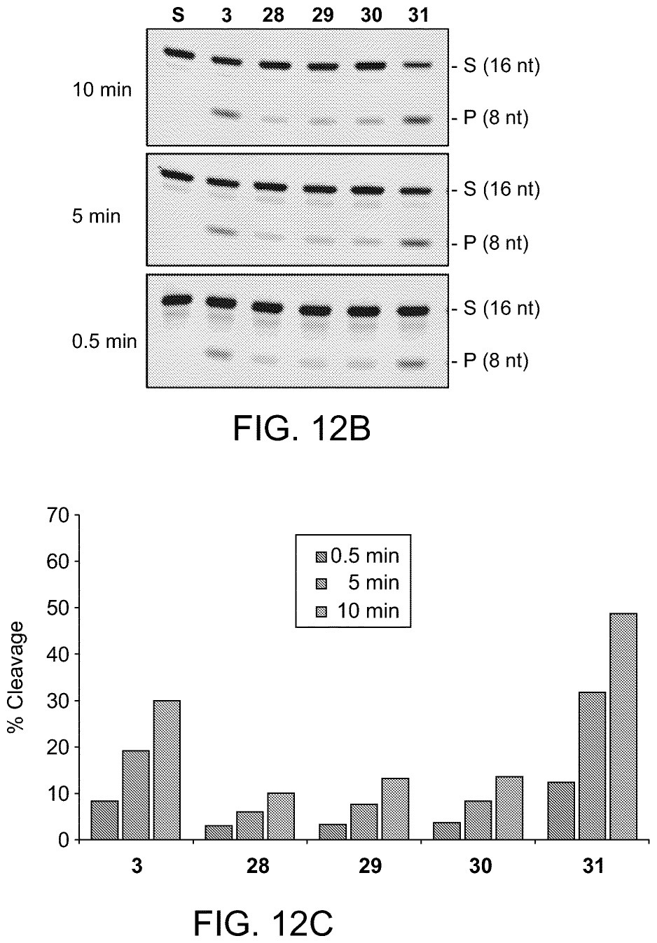

Building from our initial findings, we continued our optimization of Dz 10-23 through modification of the binding arms (FIG. 10) as well as a stepwise process of identifying critical residues in the catalytic domain. Although this sequence represents an evolutionary optimum where genetic mutations often lead to variants with reduced catalytic activity (24), very little is known about the functional consequences of chemical substitutions made to the sugar moiety. To investigate such changes, we systematically replaced each residue in the catalytic core with the corresponding OMe nucleotide (for example, replacing dA with OMe-A). The catalytic profile of the 15 variants uncovered several residues where OMe substitutions led to favorable improvements in catalytic activity (FIG. 11). Subsequent evaluation of positions C7, T8, and G14 with various combinations of OMe and MOE chemistry revealed a clear preference for OMe residues at positions 7 and 8, and a MOE residue at position 14 (FIGS. 12-14). The strong preference for OMe at positions 7 and 8 is consistent with the location of a tight turn in the folded structure of the catalytically active conformation (FIG. 7), which lies in close proximity to metal-ion binding site 1122. Augmenting the triply modified catalytic loop with additional modifications to the binding arms as well as the strategic positioning of 2 phosphorothioate linkages 11 resulted in the generation of 3 highly optimized DNAzyme constructs (variants 38, 42, and 46, FIGS. 10, 15 and 16) that are kinetically indistinguishable in RNA cleavage assays performed with the 10-fold excess substrate (FIGS. 17 and 18).

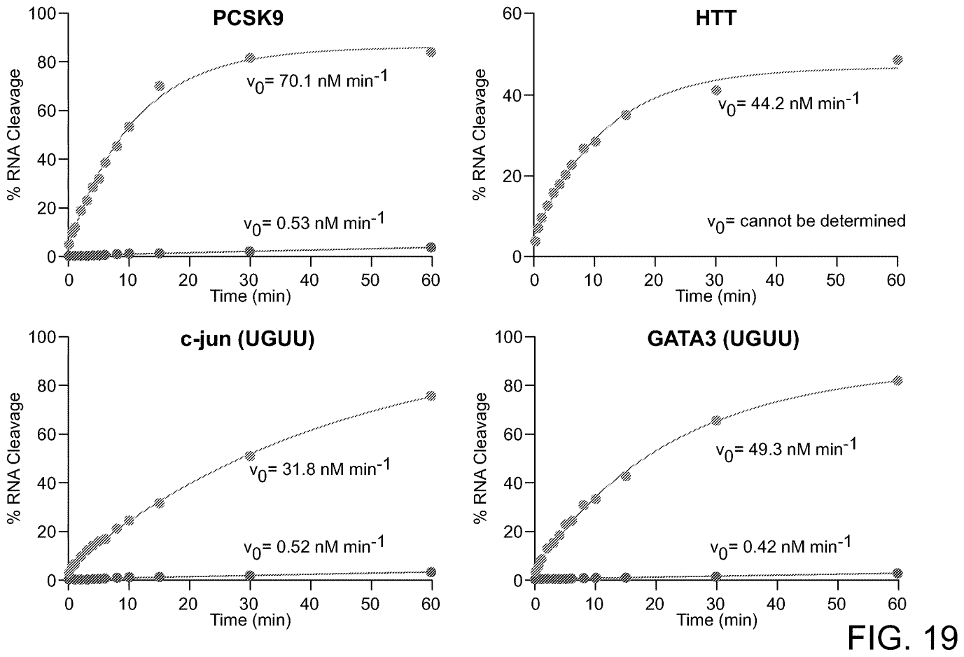

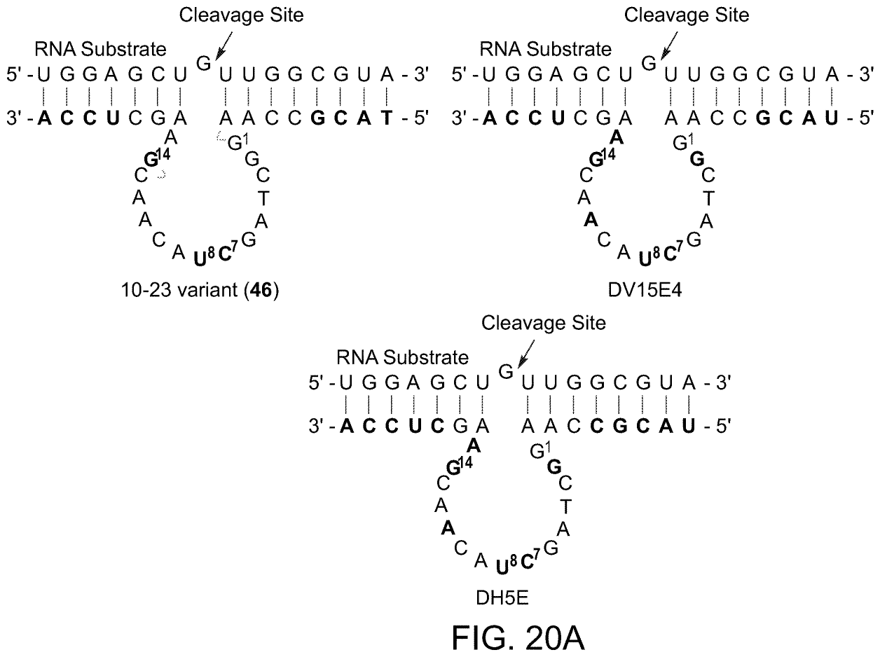

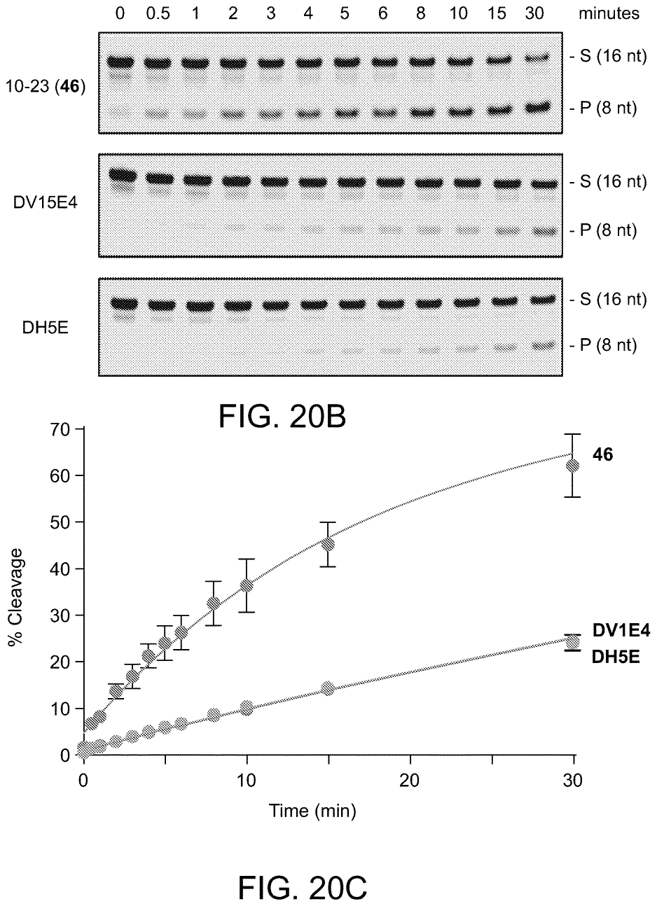

To better quantify the activity of our best 10-23 variants, we repeated our kinetic analysis under more stringent conditions in which the reaction mixture contained 100-fold excess substrate. For this experiment, initial rates and catalytic turnover were measured in triplicate for 8 variants spanning the design trajectory from the original all-DNA construct (Dz 1) to the most optimized scaffold (FIG. 2, FIG. 17). The resulting data identified variant 46 (Dz 46) as our best design. This construct functions with an initial velocity of ~58 nM/min and is capable of achieving ~65 turnovers in 30 min (FIG. 2). To the best of our knowledge, this level of activity has not been witnessed by any previous RNA-cleaving DNAzyme tested under physiological conditions and compares favorably to the unmodified parent sequence under forcing conditions of 50 mM MgCl₂ (pH 8.0) 1. Importantly, the improvements of Dz 46 over Dz 1 are also transferable to other targets (FIG. 19), indicating that it is not a sequence-specific effect. It is also more efficient than a recently described RNA-cleaving XNAzyme evaluated under equivalent conditions (46 turnovers in 96 h) 21 and more efficient than the previously best DNAzyme evaluated against the same RNA target (FIG. 20) (25).

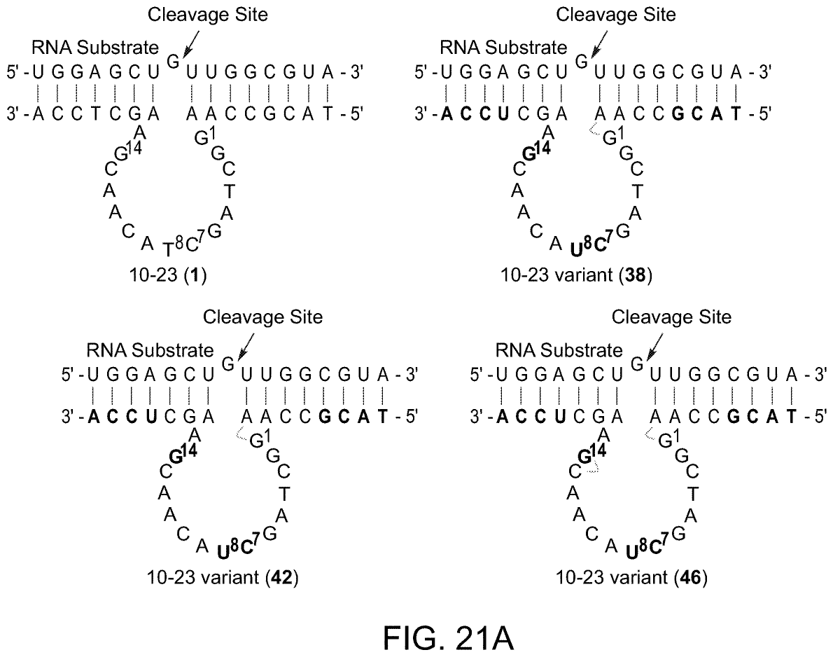

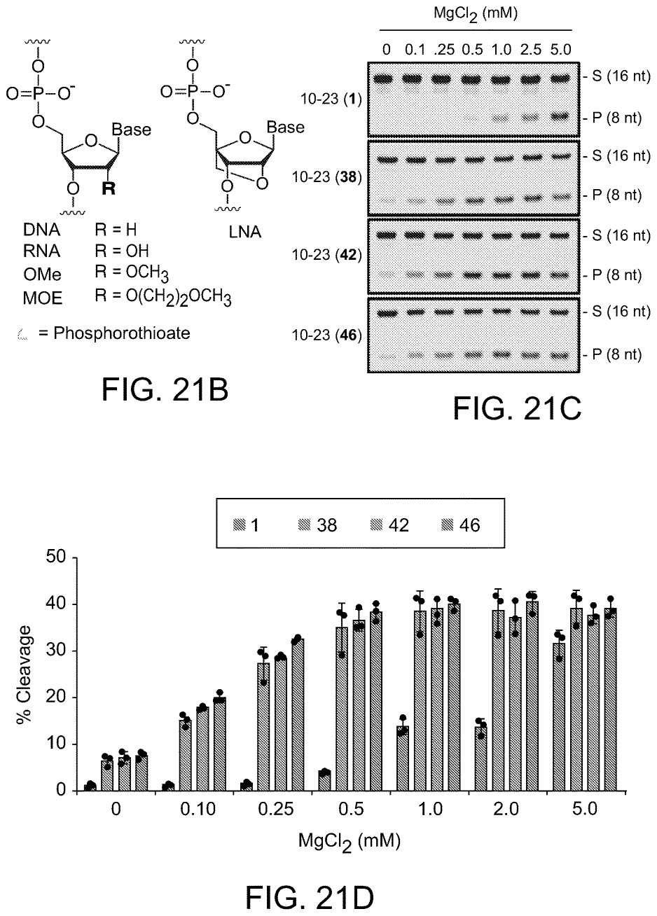

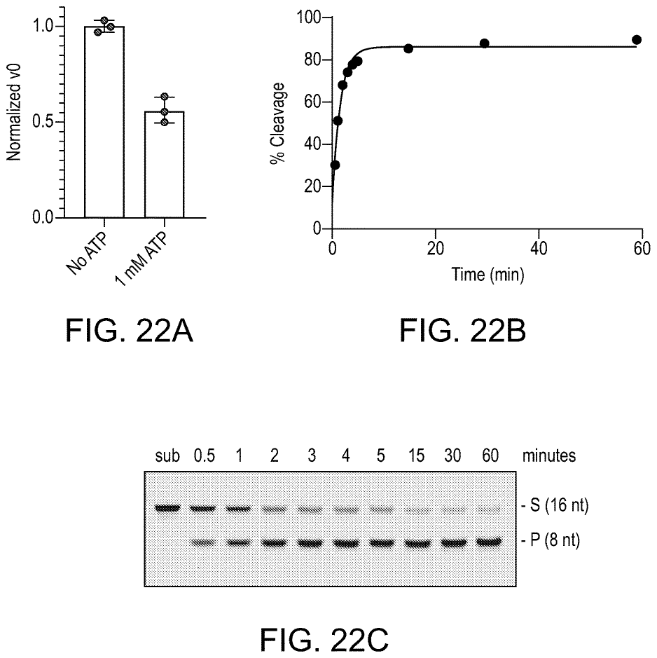

Encouraged by the dramatic improvement in multiple turnover activity observed under simulated physiological conditions, we next asked whether the chemically optimized designs functioned with reduced dependency for the divalent magnesium ion cofactor. Single-endpoint detection assays performed across a range of Mg²⁺ concentrations reveal that the best designs exhibit a strongly reduced dependency on the concentration of magnesium present in the reaction mixture than the original all DNA scaffold when tested in the cellular range of 0.1-5.0 mM MgCl₂ (FIG. 21). In fact, close concordance between the unmodified and modified Dz 10-23 versions does not occur until the reaction mixture reaches a total concentration of 5 mM MgCl₂, demonstrating that the design process gave rise to optimized scaffolds with improved magnesium coordination, which increases activity by reducing conformational instability. This observation is supported by competition assays performed in the presence of ATP, which yield only a modest 2-fold loss in activity for Dz 46 (FIG. 22) as well as pre-steady state kinetic measurements performed at 0.25 mM MgCl₂, yielding a first-order rate constant (K_{obs}) of ~0.65 min-1 for Dz 46 (FIG. 22).

### Substrate Specificity of Dz 46

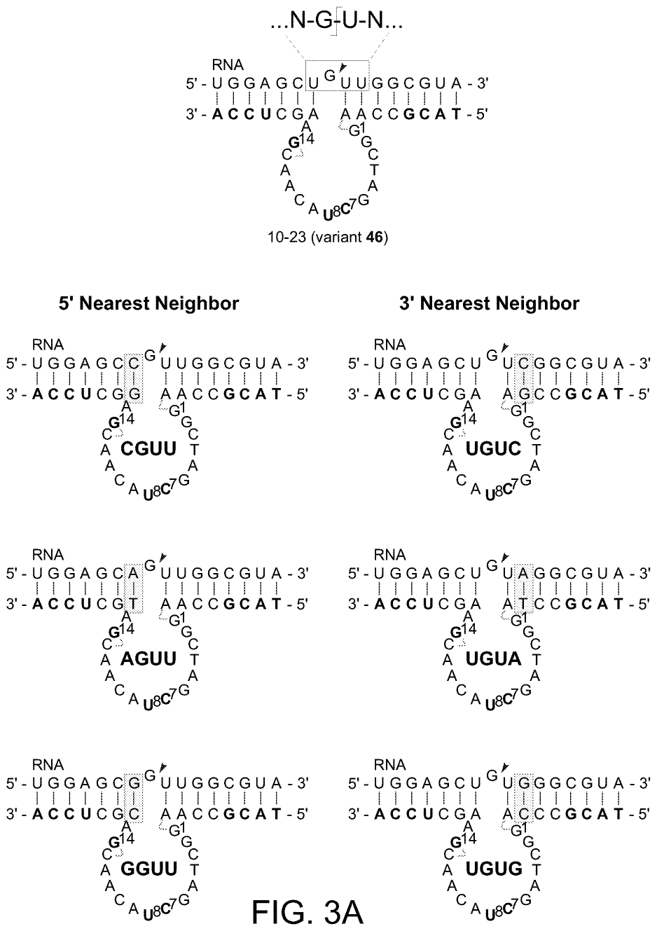

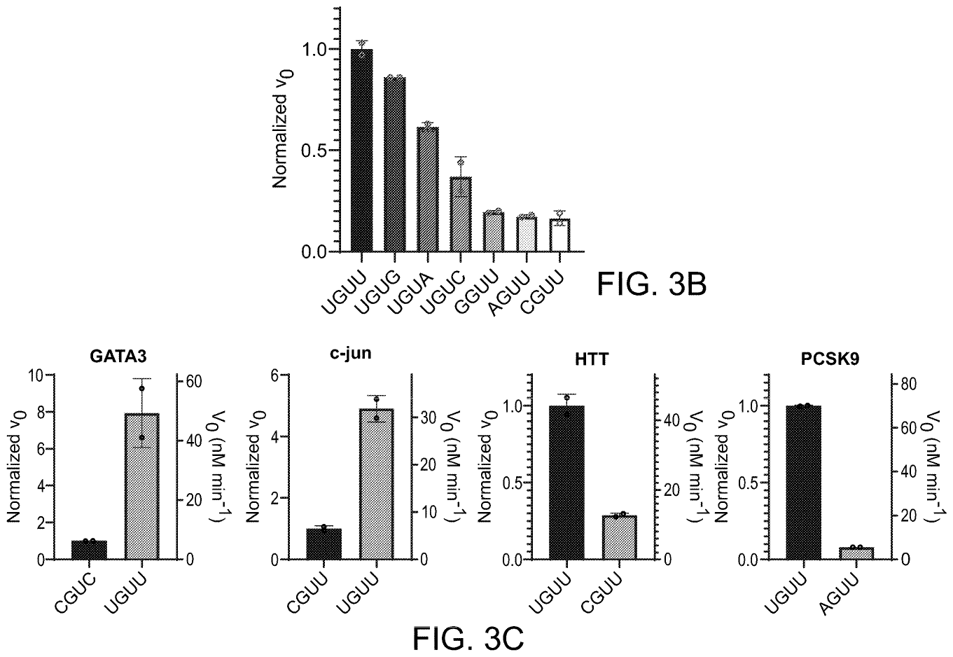

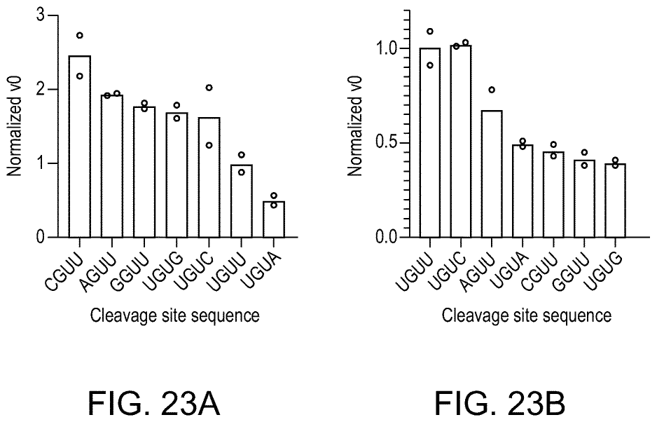

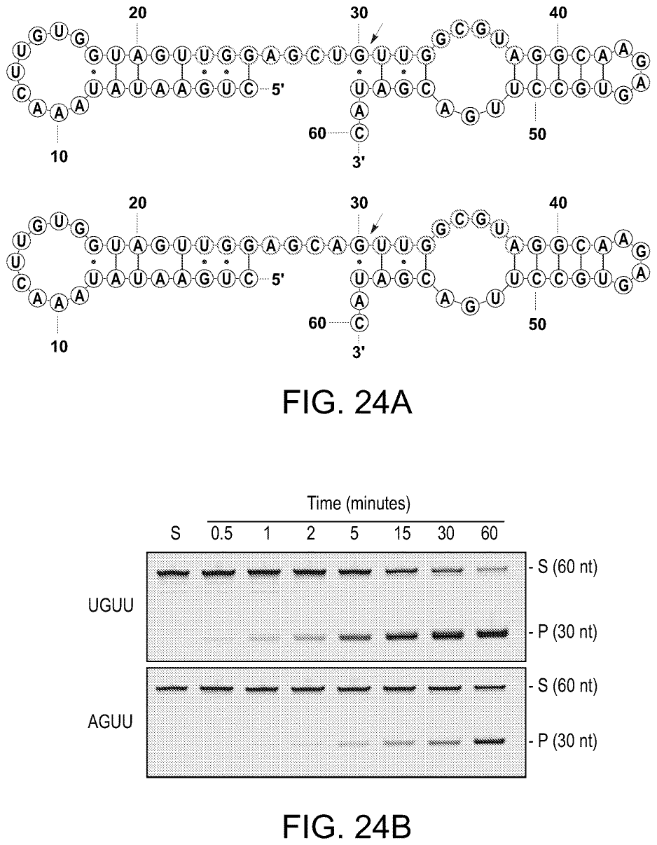

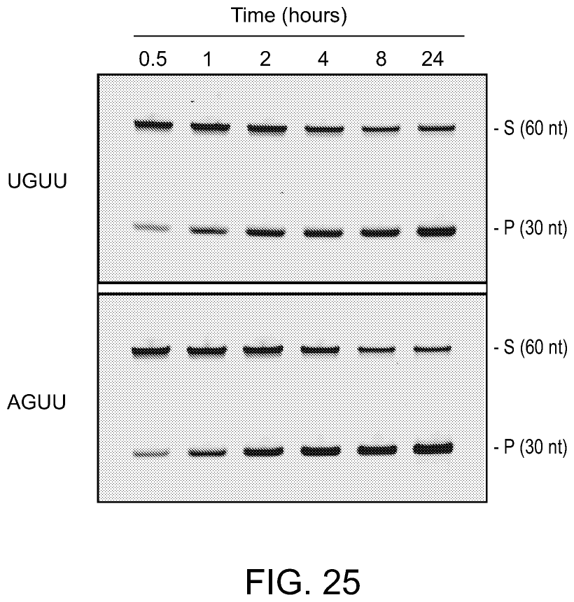

We next asked whether the chemical modifications introduced into the DNAzyme scaffold, which was intended to stabilize the catalytically active conformation of the substrate-bound DNAzyme, might favor the formation of an expanded RNA cleavage motif beyond the commonly discussed purine (R)-pyrimidine (Y) dinucleotide junction (26). Insights into this question came from the structure of the precatalytic complex of the RNA-bound DNAzyme, which show the enzyme forcing the RNA substrate to adopt a tight turn at the cleavage junction (FIG. 2) (22). To explore this question in greater detail, we systematically evaluated the nucleotide position immediately flanking the 5′ and 3′ sides of the preferred G-U cleavage motif using engineered versions of Dz 46 that were programmed to recognize RNA substrates (FIG. 3A) carrying single-nucleotide sequence alterations on the 5′ or 3′ sides of the cleavage junction. Analysis of the initial rates measured for each DNAzyme-substrate pair (FIG. 3B) uncovered a clear preference for a 5′-UGUD-3′ motif (cut site underlined and D corresponds to residues A, G, or U) that was up to 5-fold more active than 5′-XGUU-3′ motifs, where X corresponds to a G, A, or C residue. By comparison, unmodified DNAzymes (Dz 1 variant) exhibit a similar trend when assayed at 5 mM MgCl₂ but show a diminished cleavage site preference for reactions performed at 1 mM MgCl₂ (FIG. 23). Importantly, the differences observed between Dz 46-mediated cleavage of the preferred and unpreferred cleavage motifs in short size-matched RNA substrates were maintained in longer RNA substrates (FIG. 24), indicating that the trend is not limited to shorter synthetic RNA substrates. However, this trend was not observed for unmodified DNAzymes (Dz 1) against the longer RNA substrates, even at 5 mM MgCl₂ (FIG. 25). As such, these results demonstrate the potential for chemically modified DNAzymes to invade RNA secondary structures that are more likely to arise in the cell.

We validated the positive and negative predictive capabilities of the preferred cleavage motif by evaluating a diverse set of DNAzyme targets. As a test for positive predictive capability, we chose the same GATA-3 (CGUC) (27) and c-jun (CGUU) (28) cut sites previously evaluated in clinical trials as treatments for allergic asthma and nodular basal-cell carcinoma, respectively. Using appropriately designed versions of Dz 46 and short synthetic RNA targets, we compared the cleavage activity of the known cut site to engineered versions carrying the preferred UGUU cleavage motif (FIG. 3C). In both cases, sequence-matched DNAzymes targeting the preferred cut site function with higher activity (5-7-fold) than equivalent DNAzyme-substrate pairs targeting the known cut sites. Likewise, negative predictive capability assays reveal that DNAzymes targeting previously untargeted RNA cut sites identified in Huntington's (HTT, UGUU) and proprotein convertase subtilisin/kexin type 9 (29) (PCSK9, UGUU) targets outperform (3-12-fold) engineered cut sites predicted to function with suboptimal activity. The ability to identify optimal G-U cut sites within a disease-associated mRNA target should accelerate future drug discovery efforts with DNAzyme reagents by reducing the number of possible cut sites within a given target and increasing the activity of DNAzymes against desired target sites that reside in a favorable UGUD sequence context.

### Autonomous RNA Cleavage

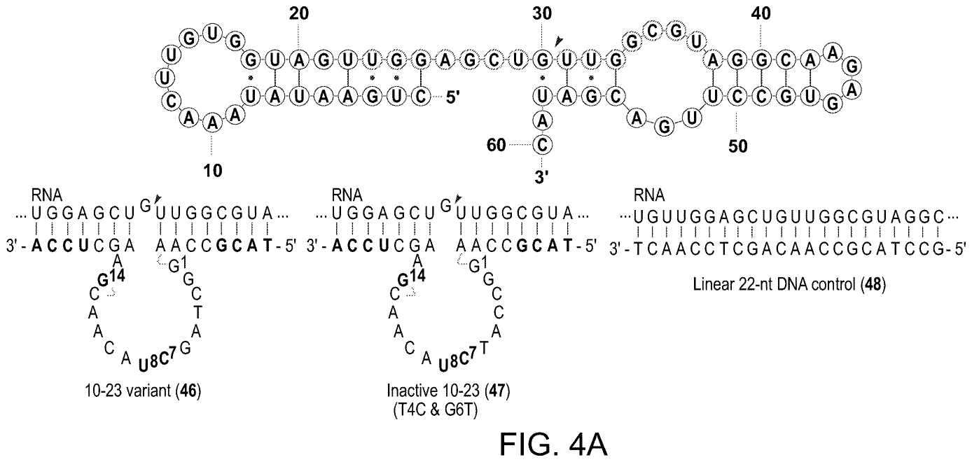

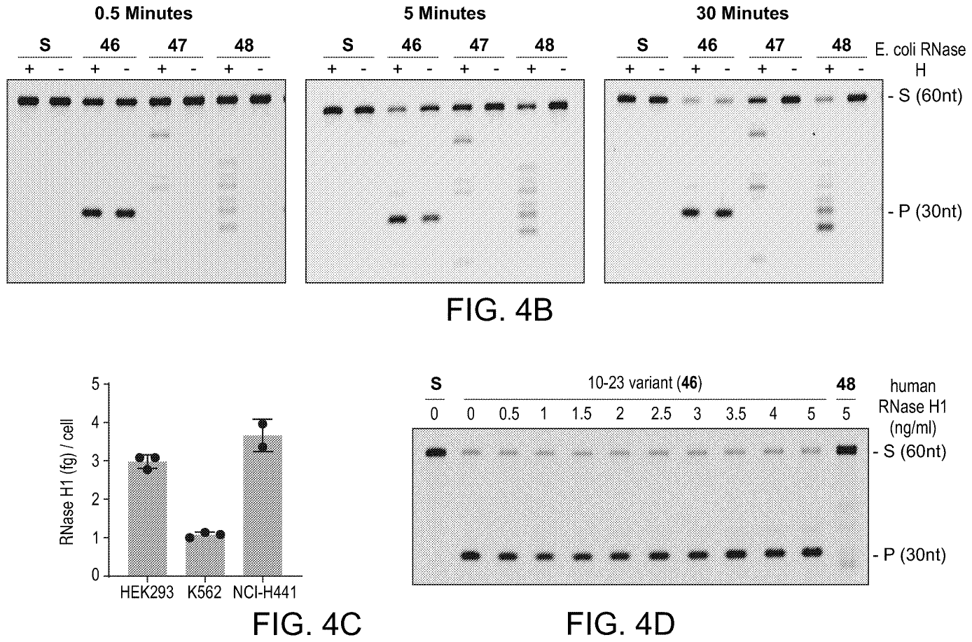

Previous studies have shown that RNase H1 contributes to RNA degradation in cultured mammalian cells transfected with unmodified DNAzymes (30). In such cases, competition exists between the intrinsic activity of the DNAzyme and cellular RNase H1, which recognizes the substrate binding arms of the DNAzyme as antisense oligonucleotides. Although chemical modifications have reduced RNase recognition (17), this topic continues to be discussed by DNAzyme development laboratories (31). We postulated that the chemical modifications introduced into the binding arms of the 10-23 scaffold, coupled with the substantial multiple-turnover activity observed under near-physiological conditions, would render Dz 46 less susceptible to the effects of an RNase H-induced cleavage mechanism. To investigate this possibility, we evaluated the catalytic activity of Dz 46 against a synthetic 60 nt RNA substrate in time-course experiments that either contained or lacked recombinant E. coli RNase HI (FIG. 4A). Analysis of the resulting denaturing PAGE gels indicate that the reaction produces the desired site-specific cleavage product regardless of whether RNase H is present or absent from the reaction mixture (FIG. 4B). By contrast, control experiments performed using a catalytically inactive version of Dz 46 and a linear antisense DNA oligonucleotide (ASO) yield cleavage patterns that are dependent upon the presence of RNase HI. The striking difference in behavior between the active DNAzyme and the control reagents indicates that Dz 46 is sufficient and necessary to produce the predicted RNA cleavage product, supporting the hypothesis that Dz 46 functions by an autonomous site-specific DNAzyme-mediated RNA cleavage mechanism.

Since our initial assays were performed using the manufacturer-recommended concentration of 0.05 units/μL of E. coli RNase HI, which is presumably required to facilitate efficient molecular biology transformations, we chose to examine the mechanism of DNAzyme cleavage under conditions that were more biologically relevant. We began by quantifying the cellular concentration of RNase H1 in cultured HEK293, K562, and NCI-H441 cell lines. Using an ELISA assay, we found that the cellular concentration of RNase H1 ranged from 1.0 to 3.5 fg/cell (FIG. 4C), which translates to ~0.5-2.5 ng/μL (Table 1). Relative to the linear DNA control, DNAzyme-mediated RNA cleavage assays performed in the physiological range of human RNase H1 produce the desired site-specific cleavage product (FIG. 4D). This observation strengthens the hypothesis that Dz 46 functions as an autonomous gene silencing reagent independent of RNase H1 engagement.

TABLE 1
HsRNAse H1 level in human cell lines measured by ELISA

| | HEK cells | K562 cells |
|---|---|---|
| Diameter (μm) | 13.9 ± 0.13^{a} | 20.94 ± 1.08^{b} |
| Cell volume (μm³) | 1405.47 | 4805.17 |
| Cell volume (pL) | 1.40547 | 4.80517 |
| Amt HsRnase H1/cell by ELISA (fg) | 2.98 | 1.07 |
| [Rnase H1]/cell (fg/pL) | 2.12 | 0.22 |
| [Rnase H1]/cell (ng/μL) | 2.12 | 0.22 |
| HsRNase H1 MW (Kg/mol) | 37.60 | 37.60 |
| [HsRNaseH1] (nM) | 56.37 | 5.93 |

^{a}Mateus, A, et al., (2013) Mol. Pharmaceutics, 10: 2467-2478; BNID: 108893
^{b}Jiang, Xiaoyu et al., (2016) Magnetic Resonance in Medicine, 75: 2076-1085

### Allele-Specific RNA Cleavage

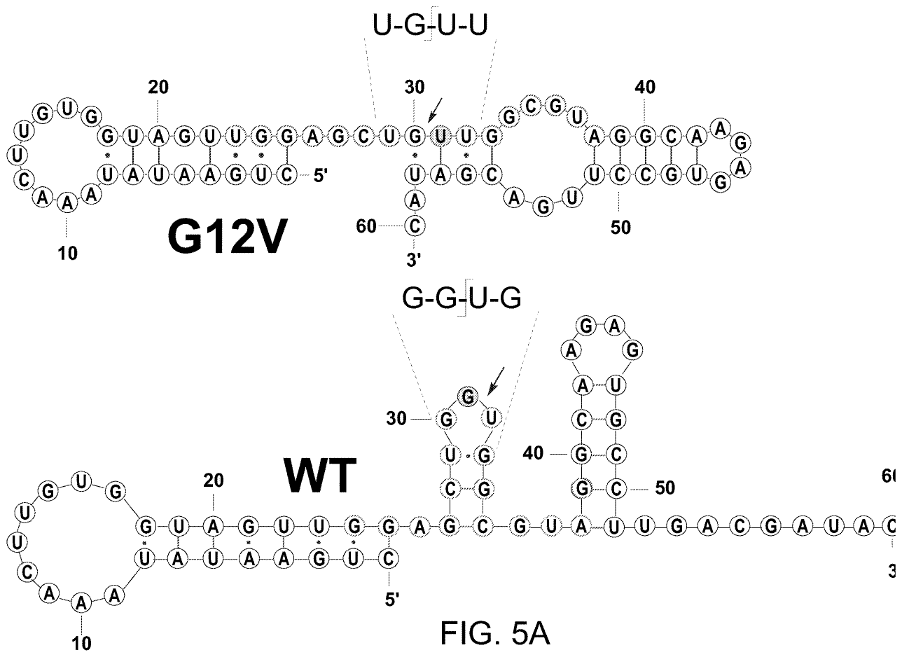

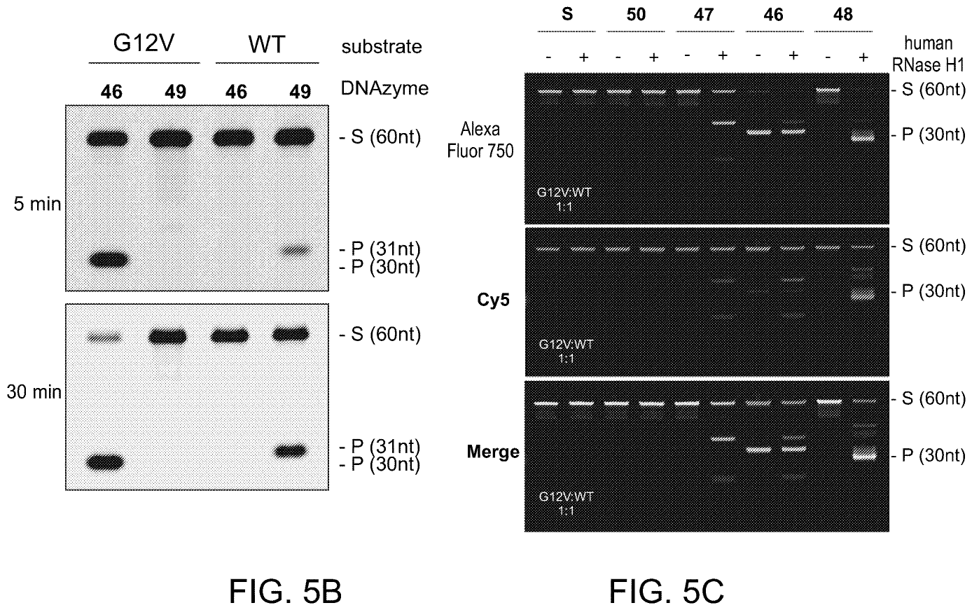

In contrast to protein-based gene silencing technologies, the mechanism of DNAzyme-mediate strand cleavage allows for the precise targeting of specific nucleotide mutations (SNPs) responsible for disease-associated phenotypes (2). A classic example is KRAS—a master regulator of cell growth and division with activator mutations found in ~25% of all human cancers (32). One commonly studied KRAS mutation is G12V, caused by a G→U mutation in position 2 of codon 12, which leads to glycine (G) to valine (V) mutation in the amino acid sequence (33). Unlike G12C, the G12V mutation is viewed as an undruggable target as the valine residue lacks a nucleophilic moiety for targeting by small molecule inhibitors (34,35). We assessed the ability of Dz 46 to distinguish allelic mutations in synthetic KRAS RNA substrates represented by a 60 nt segment of the mRNA (FIG. 5A). DNAzymes engineered to recognize the wild-type and mutant KRAS substrates functioned with >99% specificity (FIG. 5B), as no cross-reactivity was observed for either DNAzyme against the opposing substrate. Consistent with our substrate specificity study, Dz 46 is ~5-fold more active than Dz 49, which is due to the presence of a favorable UGUU cleavage motif in the mutant substrate and a less favorable GGUG motif in the wild-type substrate.

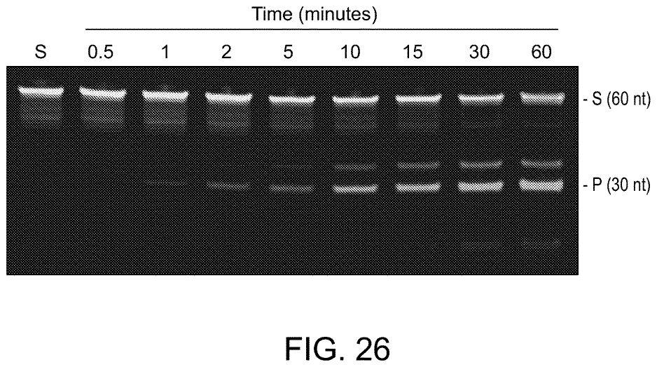

Next, we examined the potential for allele-specific strand cleavage under competitive conditions in which both allelic substrates are present in the reaction mixture. This assay is more representative of the cellular environment and includes reactions that were performed in the presence and absence of human RNase H1 poised at a concentration that is 2-fold above the cellular range. To distinguish the wild-type and mutant substrates, the synthetic RNA strands were prepared with 5′-modified Cy5 (red) and Alexa Fluor 750 (green) tags, respectively. In these assays, Dz 46 exclusively cleaves the mutant KRAS substrate; however, a trace amount of RNase H1-mediated cleavage is observed for the wild-type substrate, which is attributed to the formation of a stable but inactive complex between the mutant Dz and wild-type substrate (FIG. 5C and FIG. 26). Standard positive controls performed with inactive DNAzyme and the ASO yield the expected RNase H1 cleavage products, while the non-binding DNAzyme and substrate only controls show no signs of RNA cleavage (FIG. 5C).

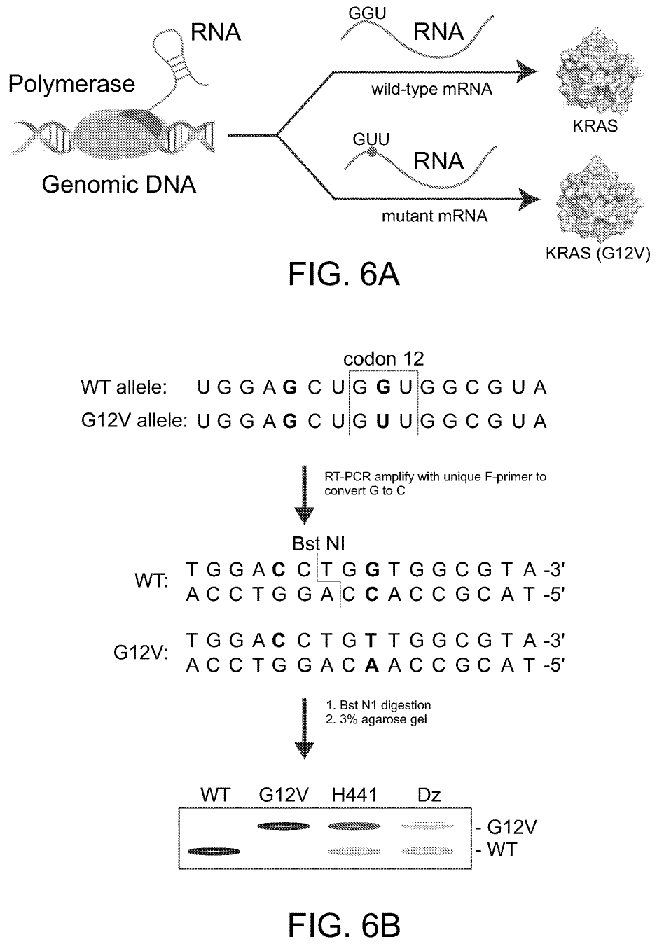

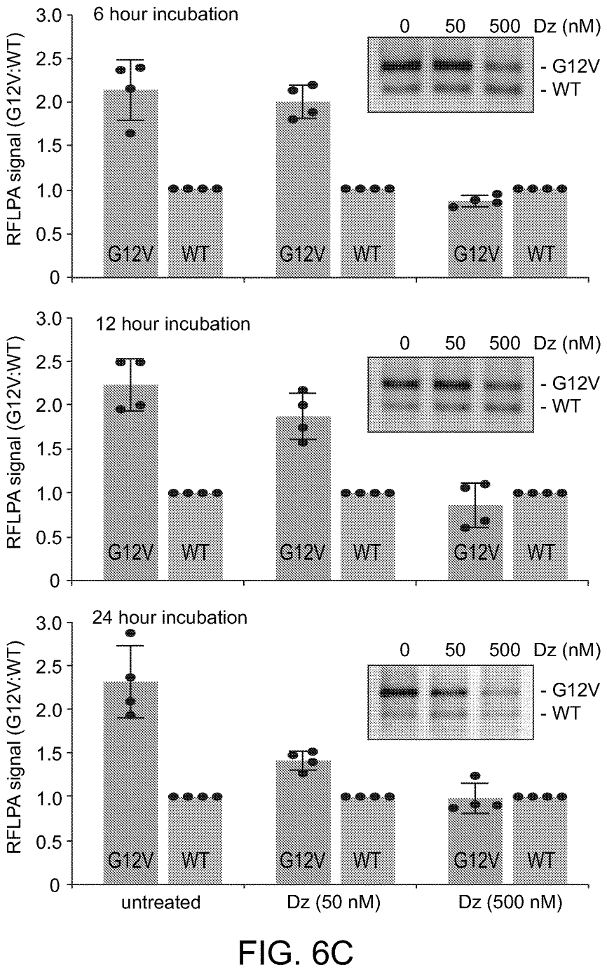

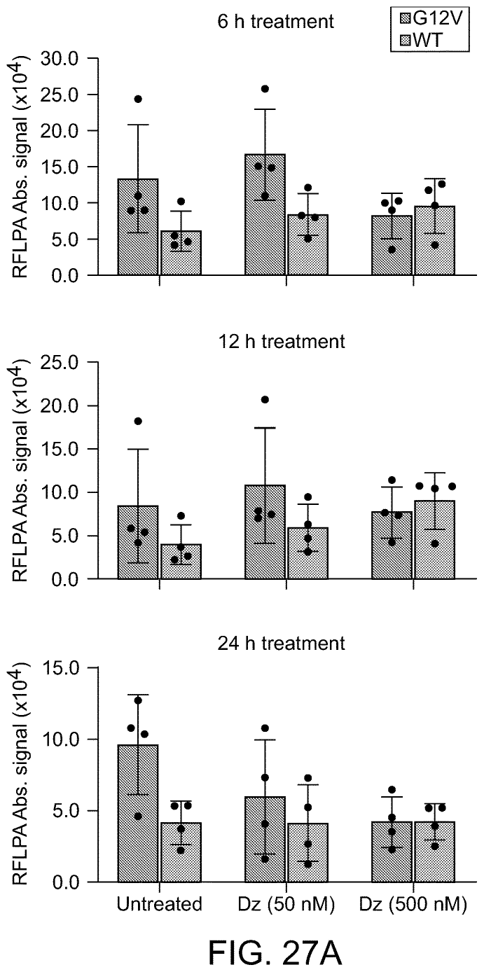

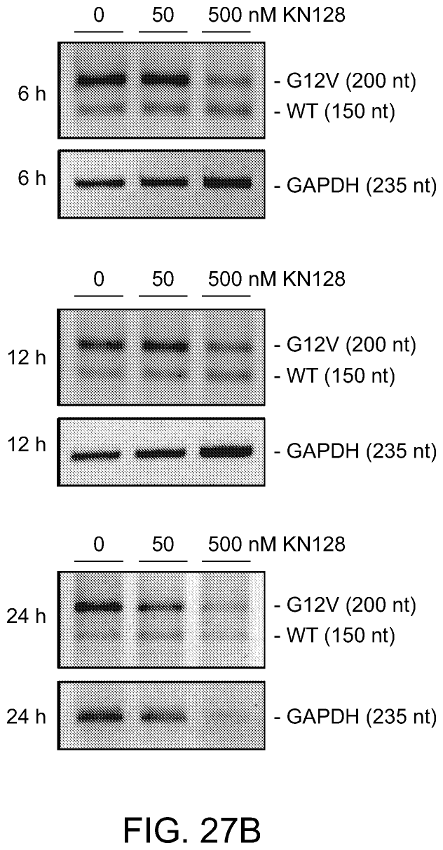

Having demonstrated that Dz 46 is capable of allele-specific RNA cleavage, we next asked whether similar effects could be achieved for a high-value endogenous mRNA target. For this study, we chose NCI-H441 as a model adenocarcinoma cell line expressing 3-4 genomic copies of G12V to wild-type KRAS per cell (FIG. 6A). We used a PCR restriction fragment length polymorphism assay (PCR-RFLP) to distinguish cleavage between the two allelic mRNA transcripts (FIG. 6B) (36). With this technique, RT-PCR is used to introduce a mutation into the amplicons that lead to the formation of a restriction site in the wild-type sequence only. Following restriction enzyme digestion, allele-specific gene knockdown activity is assessed by quantifying the ratio of the mutant and wild-type bands by agarose gel electrophoresis. Our results indicate that modest mRNA knockdown of the mutant gene is observed after 24 h of treatment with 50 nM DNAzyme (FIG. 6C). Higher dosing with 500 nM DNAzyme leads to rapid and sustained knockdown of mutant KRAS G12V mRNA, as observed between 6 and 24 h of treatment (FIG. 6C, FIG. 27). This result compares favorably against that of X10-23 targeting the same G12V mutation, which required 96 h of treatment to observe similar levels of allele-specific gene silencing (37).

The site-specific cleavage of a target RNA molecule by a natural or in vitro evolved nucleic acid enzyme is one of the best-characterized reactions in nucleic acid chemistry (38,39). In fact, the mechanism of action and strategy for evolving these reagents is so well understood that this functional paradigm has been extended to XNAzymes, whose backbone structures encompass synthetic forms of nucleic acid polymers composed entirely of non-natural sugar-phosphate linkages (19,20,21). However, despite major growth in this area of synthetic biology, the ability to generate nucleic acid enzymes with robust multiple turnover activity under physiological conditions has proven more challenging than expected. Even Darwinian evolution experiments aimed at querying large combinatorial libraries for individual sequences that function with reduced magnesium dependency have fallen short of expectations (13). Given the significant effort expended toward this problem, one could conclude that the chemical repertoire available to natural genetic polymers is insufficient to achieve the level of activity required for clinical applications.

In this Example, we evaluated the potential for chemical evolution to overcome this barrier by augmenting a known DNAzyme with additional functional properties. We postulated that the substitution of natural DNA residues for synthetic congeners at key structural positions in a DNAzyme scaffold might favor the formation of a catalytically active conformation by shifting the equilibrium of folded structures away from the ensemble of alternative yet unproductive conformations. Through iterative cycles of design, we uncovered permissive sites in the 10-23 DNAzyme scaffold where chemical modifications afforded clear stepwise improvements in multiple turnover activity under near-physiological conditions. This effort culminated in the discovery of Dz 46, a chemically engineered 10-23 variant that functions with unprecedented multiple turnover activity under experimental conditions simulating the cellular environment. Biochemical characterization indicates that the heavily modified DNAzyme achieves heightened catalytic activity through improved cofactor binding or coordination, which is required for proper folding and function of the DNAzyme. We suggest that the approach taken was sufficiently general that it could be applied to other DNAzymes, especially those whose structures have been determined. While insights from the NMR-based structure of 10-23 helped to guide the design of Dz46, other structural insights from RNA-cleaving nucleic acid enzymes that share a common active site motif known as the L-platform, may facilitate the design of other DNAzymes, such as 8-17 (40). It should also be noted that RNA and DNA-catalyzed RNA-cleavage mechanisms described by Breaker and others may shed light on other potential design principles for engineered DNAzymes (41).

One interesting result to come from this study was the finding that Dz 46 favors a highly specific tetranucleotide cleavage motif (UGUD). This observation is likely due to the increased structural rigidity of the DNAzyme scaffold caused by the introduction of diverse chemical modifications throughout the catalytic loop and binding arms of the molecule. Elucidation of the preferred cleavage motif, which was validated using both positive and negative predictive agreement assays across four different disease-associated targets, offers a streamlined approach for designing new 10-23 variants for clinical applications as it reduces the number of G-U cut sites in an mRNA target to a subset that reside in a preferred sequence context. We note, however, that the additional constraints imposed by the preferred cleavage motif could restrict the number of targets amenable to Dz cleavage. Another striking feature of our study was the discovery that Dz 46 functions as an autonomous reagent under physiologically relevant concentrations of RNase H1, which were empirically determined using an ELISA assay. We postulate that the fast catalytic rate, coupled with the presence of chemical modifications in the substrate binding arms, greatly reduces the potential for RNase H1 engagement.

One area of biomedicine where DNAzymes, like Dz 46, could have a major impact on human health is in the validation and treatment of allele-specific genetic diseases (42). Currently, protein-based gene silencing reagents struggle to distinguish SNPs in an RNA target (43,44), while DNAzymes can unequivocally target mutant and wild-type alleles with high efficiency and precision. In this way, Dz 46 offers an additional tool in the medicinal chemistry toolbox that allows for the precision cutting of an allelic mRNA target in cultured mammalian cells. Recognizing that >200,000 known acquired GU mutations can be found in the Cancer Genome Atlas database 37, it is reasonable to assume that Dz 46 is well poised to expand the concept of allele-specific gene silencing.

In summary, we applied a chemical evolution approach to discover a highly modified 10-23 DNAzyme variant that functions with unusually high multiple turnover activity under near-physiological conditions. The generality of our approach opens the door to other examples of DNAzymes that could be improved for future biomedical applications.

### REFERENCES

1. Santoro, S. W. & Joyce, G. F. Proc. Natl Acad. Sci. USA 94, 4262-4266 (1997).
2. Santoro, S. W. & Joyce, G. F. Biochemistry 37, 13330-13342 (1998).
3. Joyce, G. F. Methods Enzymol. 341, 503-517 (2001).
4. Fokina, A. A., et al. Expert Opin. Biol. Ther. 15, 689-711 (2015).
5. Victor, J., et al. J. 47, 333-343 (2018).
6. Wang, Y., et al. Chem. Sci. 9, 1813-1821 (2018).
7. Khvorova, A. & Watts, J. K. Nat. Biotechnol. 35, 238-248 (2017).
8. Bennett, C. F. Annu. Rev. Med. 70, 307-321 (2019).
9. Fokina, A. A., et al. Expert. Opin. Drug Deliv. 14, 1077-1089 (2017).
10. Li, Y., et al. Bioorg. Med. Chem. Lett. 62, 128633 (2022).
11. Nawrot, B. et al. FEBS J. 274, 1062-1072 (2007).
12. Takahashi, H. et al. FEBS Lett. 560, 69-74 (2004).
13. Malik, T. N. & Chaput, J. C. Curr. Res. Chem. Biol. 1, 100012 (2021).
14. Fokina, A. A., et al. Biochemistry 51, 2181-2191 (2012).
15. Vester, B. et al. J. Am. Chem. Soc. 124, 13682-13683 (2002).
16. Damha, M. J. et al. J. Am. Chem. Soc. 120, 12976-12977 (1998).
17. Wang, Y., et al., Chem. 13, 319-326 (2021).
18. Nikoomanzar, A., et al. Q. Rev. Biophys. 53, e8 (2020).
19. Wang, Y., et al. Nat. Commun. 9, 5067 (2018).
20. Wang, Y. et al. Nat. Chem. 14, 350-359 (2022).
21. Taylor, A. I., et al. Nat. Chem. 14, 1295-1305 (2022).
22. Borggrafe, J. et al. Nature 601, 144-149 (2022).
23. Cornish-Bowden, A. et al. Nucleic Acids Res. 13, 3021-3030 (1985).
24. Zaborowska, Z., et al. J. Biol. Chem. 277, 40617-40622 (2002).
25. Schubert, S. et al. Nucleic Acids Res. 31, 5982-5992 (2003).
26. Cruz, R. P., et al. Chem. Biol. 11, 57-67 (2004).
27. Krug, N. et al. N. Engl. J. Med. 372, 1987-1995 (2015).
28. Cho, E. A. et al. Lancet 381, 1835-1843 (2013).
29. Jin, J. et al. J. Am. Med. Assoc. 314, 2320 (2015).
30. Young, D. D., et al. J. Am. Chem. Soc. 132, 6183-6193 (2010).
31. Spitale, R. C. & Chaput, J. C. Nat. Chem. 14, 859-861 (2022).
32. McCormick, F. Clin. Cancer Res. 21, 1797-1801 (2015).
33. Chiosea, S. I., et al. Mod. Pathol. 24, 1571-1577 (2011).
34. Janes, M. R. et al. Cell 172, 578-589.e517 (2018).
35. Canon, J. et al. Nature 575, 217-223 (2019).
36. Sunaga, N. et al. Mol. Cancer Ther. 10, 336-346 (2011).
37. Nguyen, K., et al. J. Am. Chem. Soc. 143, 4519-4523 (2021).
38. Wilson, D. S. & Szostak, J. W. Annu. Rev. Biochem. 68, 611-647 (1999).
39. Narlikar, G. J. & Herschlag, D. Annu. Rev. Biochem. 66, 19-59 (1997).
40. Gaines, C. S., et al. RNA 26, 111-125 (2020).
41. Breaker, R. R. et al. RNA 9, 949-957 (2003).
42. Kim, J. et al. N. Engl. J. Med. 381, 1644-1652 (2019).
43. Liao, H. et al. PLOS ONE 6, e28582 (2011).
44. Christie, K. A. et al. Sci. Rep. 7, 16174 (2017).
45. Sau, S. P., et al. J. Org. Chem. 81, 2302-2307 (2016).

Throughout this application various publications are referenced. The disclosures of these publications in their entireties are hereby incorporated by reference into this application in order to describe more fully the state of the art to which this invention pertains.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

1. A nucleotide construct comprising:
(a) a 3′ substrate binding arm;
(b) a 5′ substrate binding arm; and
(c) a catalytic domain having the nucleic acid sequence of positions 1-15 of SEQ ID NO: 1 disposed between the 3′ substrate binding arm and the 5′ substrate binding arm;
wherein the 3′ substrate binding arm and the 5′ substrate binding arm are complementary to nucleotides flanking a G-U cleavage site in a target RNA, wherein the first nucleotide 3′ of the catalytic domain is an adenosine (A), and wherein the second nucleotide 5′ of the catalytic domain is an A or a pyrimidine (Y).

2. The nucleotide construct of claim 1, wherein the 3′ substrate binding arm and the 5′ substrate binding arm comprise one or more modified nucleic acids.

3. The nucleotide construct of claim 2, wherein the modified nucleic acids comprise a 2′-O-methoxyethylribonucleic acid (MOE), 2′-O-methylribonucleic acid (OMe), locked nucleic acid (LNA), threose nucleic acid (TNA), MNA, SNA, GNA, HNA, diaminopurine (DAP), C-5 modified pyrimidines, pseudo uridicine, or C-5-methyl deoxycytidine.

4. The nucleotide construct of claim 2, wherein a first modified nucleic acid is disposed at the terminus of the 3′ substrate binding arm, a second modified nucleic acid is disposed at the terminus of the 5′ substrate binding arm, and three OMe modifications are positioned adjacent to each of the modified nucleic acids.

5. The nucleotide construct of claim 1, wherein the 3′ substrate binding arm and the 5′ substrate binding arm are each at least 5 nucleotides in length.

6. The nucleotide construct of claim 1, wherein the 3′ substrate binding arm and the 5′ substrate binding arm are each 6 to 10 nucleotides in length.

7. The nucleotide construct of claim 1, wherein the target RNA is KRAS mRNA.

8. The nucleotide construct of claim 1, wherein the dC at position 7 and the dT at position 8 are each substituted with a OMe to form an OMe-C and an OMe-U, respectively, wherein the dG at position 14 is substituted with an OMe or a MOE residue to form an OMe-G or an MOE-G, and wherein a first phosphorothioate linkage is disposed between positions 0 and 1.

9. A method of cleaving a target mRNA sequence, the method comprising contacting the target mRNA sequence with a nucleotide construct of claim 1.

10. The method of claim 9, wherein the cleavage activity is enhanced at least two-fold relative to cleavage using a construct lacking an A as the first nucleotide 3′ of the catalytic domain and an A or Y as the second nucleotide 5′ of the catalytic domain.

11. A method of modulating the translation of a disease-associated protein, the method comprising contacting a target mRNA sequence that encodes the disease-associated protein with a nucleotide construct of claim 1.

12. A method of detecting the presence of a target RNA sequence in a sample, the method comprising:
(a) contacting the sample with a nucleotide construct of claim 1, wherein the 3′ substrate binding arm and the 5′ substrate binding arm of the nucleotide construct are complementary to the target RNA sequence; and
(b) measuring cleavage of the target RNA sequence, wherein cleavage is indicative of presence of the target RNA sequence in the sample.

13. The method of claim 12 wherein the nucleotide construct is modified with a marker that is detectable upon cleavage, and wherein the measuring comprises measuring the presence of the detectable marker.

14. The method of claim 13, wherein the detectable marker is a fluorophore.


SEQUENCE LISTING (47 sequences)

SEQ ID NO: 1 - DNA, 15 bp, synthetic construct
  source 1..15: mol_type = other DNA
ggctagctacaacga

SEQ ID NO: 2 - DNA, 30 bp, synthetic construct
  source 1..30: mol_type = other DNA
  misc_feature 1..15: note = DNA
  modified_base 16: mod_base = um
  misc_feature 17..26: note = DNA
  modified_base 27: mod_base = um
  misc_feature 28..30: note = DNA
  modified_base 1: mod_base = OTHER; note = Locked nucleic acid
  modified_base 15: mod_base = cm
  modified_base 28..29: mod_base = cm
  modified_base 22: mod_base = OTHER; note = 2'-O-methoxyethyl
  modified_base 30: mod_base = OTHER; note = Locked nucleic acid
  modified_base 3: mod_base = cm
  modified_base 4: mod_base = gm
  modified_base 2: mod_base = OTHER; note = 2'-O-Methyladenosine
  misc_feature 16: note = RNA
  misc_feature 27: note = RNA
tacgccaaggctagctacaacgaagctcca

SEQ ID NO: 3 - DNA, 30 bp, synthetic construct; identical to SEQ ID NO: 2
  source 1..30: mol_type = other DNA
  modified_base 1: mod_base = OTHER; note = Locked nucleic acid
  modified_base 30: mod_base = OTHER; note = Locked nucleic acid
  misc_feature 1..15: note = DNA
  misc_feature 16: note = RNA
  misc_feature 17..26: note = DNA
  misc_feature 27: note = RNA
  misc_feature 28..30: note = DNA
  modified_base 22: mod_base = OTHER; note = 2'-O-methoxyethyl
  modified_base 11: mod_base = OTHER; note = alpha-L-threofuranosyl cytidine
  modified_base 16: mod_base = um
  modified_base 27: mod_base = um
  modified_base 2: mod_base = OTHER; note = 2'-O-Methyladenosine
  modified_base 3: mod_base = cm
  modified_base 4: mod_base = gm
  modified_base 15: mod_base = cm
  modified_base 27: mod_base = um
  modified_base 28..29: mod_base = cm

SEQ ID NO: 4 - RNA, 16 nt, synthetic construct
  source 1..16: mol_type = other RNA
tggagctgttggcgta

SEQ ID NO: 5 - DNA, 30 bp, synthetic construct
  modified_base 16: mod_base = um
  modified_base 1: mod_base = OTHER; note = Locked nucleic acid
  modified_base 30: mod_base = OTHER; note = Locked nucleic acid
  modified_base 2: mod_base = OTHER; note = 2'-O-Methyladenosine
  modified_base 22: mod_base = OTHER; note = 2'-O-methoxyethyl
  misc_feature 1..15: note = DNA
  source 1..30: mol_type = other DNA
  misc_feature 28..30: note = DNA
  modified_base 3: mod_base = cm
  modified_base 4: mod_base = gm
  modified_base 27: mod_base = um
  misc_feature 17..26: note = DNA
  modified_base 28..29: mod_base = cm
  modified_base 24: mod_base = gm
  modified_base 15: mod_base = cm
  misc_feature 16: note = RNA
  misc_feature 27: note = RNA
tacgccaaggctagctacaacgaggctcca

SEQ ID NO: 6 - DNA, 30 bp, synthetic construct
  misc_feature 1..15: note = DNA
  modified_base 16: mod_base = um
  misc_feature 17..26: note = DNA
  modified_base 27: mod_base = um
  misc_feature 28..30: note = DNA
  source 1..30: mol_type = other DNA
  modified_base 30: mod_base = OTHER; note = Locked nucleic acid
  modified_base 1: mod_base = OTHER; note = Locked nucleic acid
  modified_base 22: mod_base = OTHER; note = 2'-O-methoxyethyl
  modified_base 2: mod_base = OTHER; note = 2'-O-Methyladenosine
  modified_base 28..29: mod_base = cm
  modified_base 4: mod_base = gm
  modified_base 3: mod_base = cm
  modified_base 15: mod_base = cm
  modified_base 24: mod_base = tm
  misc_feature 16: note = RNA
  misc_feature 27: note = RNA
tacgccaaggctagctacaacgatgctcca

SEQ ID NO: 7 - DNA, 30 bp, synthetic construct
  source 1..30: mol_type = other DNA
  modified_base 1: mod_base = OTHER; note = Locked nucleic acid
  modified_base 30: mod_base = OTHER; note = Locked nucleic acid
  modified_base 27: mod_base = um
  modified_base 22: mod_base = OTHER; note = 2'-O-methoxyethyl
  misc_feature 28..30: note = DNA
  misc_feature 1..15: note = DNA
  modified_base 2: mod_base = OTHER; note = 2'-O-Methyladenosine
  modified_base 28..29: mod_base = cm
  modified_base 4: mod_base = gm
  misc_feature 17..26: note = DNA
  modified_base 16: mod_base = um
  modified_base 3: mod_base = cm
  modified_base 15: mod_base = cm
  modified_base 24: mod_base = cm
  misc_feature 16: note = RNA
  misc_feature 27: note = RNA
tacgccaaggctagctacaacgacgctcca

SEQ ID NO: 8 - DNA, 30 bp, synthetic construct
  source 1..30: mol_type = other DNA
  misc_feature 28..30: note = DNA
  misc_feature 1..15: note = DNA
  modified_base 2: mod_base = OTHER; note = 2'-O-Methyladenosine
  misc_feature 17..26: note = DNA
  modified_base 16: mod_base = um
  modified_base 27: mod_base = um
  modified_base 15: mod_base = cm
  modified_base 3: mod_base = cm
  modified_base 4: mod_base = gm
  modified_base 22: mod_base = OTHER; note = MOE
  modified_base 28..29: mod_base = cm
  modified_base 30: mod_base = OTHER; note = Locked nucleic acid
  modified_base 1: mod_base = OTHER; note = Locked nucleic acid
  modified_base 7: mod_base = gm
  misc_feature 16: note = RNA
  misc_feature 27: note = RNA
tacgccgaggctagctacaacgaagctcca

SEQ ID NO: 9 - DNA, 30 bp, synthetic construct
  source 1..30: mol_type = other DNA
  modified_base 1: mod_base = OTHER; note = Locked nucleic acid
  modified_base 30: mod_base = OTHER; note = Locked nucleic acid
  misc_feature 1..15: note = DNA
  misc_feature 17..26: note = DNA
  misc_feature 28..30: note = DNA
  modified_base 22: mod_base = OTHER; note = 2'-O-methoxyethyl
  modified_base 2: mod_base = OTHER; note = 2'-O-Methyladenosine
  modified_base 3: mod_base = cm
  modified_base 4: mod_base = gm
  modified_base 15: mod_base = cm
  modified_base 16: mod_base = um
  modified_base 27: mod_base = um
  modified_base 28..29: mod_base = cm
  modified_base 7: mod_base = tm
  misc_feature 16: note = RNA
  misc_feature 27: note = RNA
tacgcctaggctagctacaacgaagctcca

SEQ ID NO: 10 - DNA, 30 bp, synthetic construct
  source 1..30: mol_type = other DNA
  modified_base 1: mod_base = OTHER; note = Locked nucleic acid
  modified_base 30: mod_base = OTHER; note = Locked nucleic acid
  misc_feature 1..15: note = DNA
  misc_feature 17..26: note = DNA
  misc_feature 28..30: note = DNA
  modified_base 22: mod_base = OTHER; note = 2'-O-methoxyethyl
  modified_base 2: mod_base = OTHER; note = 2'-O-Methyladenosine
  modified_base 3: mod_base = cm
  modified_base 4: mod_base = gm
  modified_base 15: mod_base = cm
  modified_base 16: mod_base = um
  modified_base 27: mod_base = um
  modified_base 7: mod_base = cm
  misc_feature 16: note = RNA
  misc_feature 27: note = RNA
  modified_base 28..29: mod_base = cm
tacgcccaggctagctacaacgaagctcca

SEQ ID NO: 11 - RNA, 16 nt, synthetic construct
  source 1..16: mol_type = other RNA
tggagccgttggcgta

SEQ ID NO: 12 - RNA, 16 nt, synthetic construct
  source 1..16: mol_type = other RNA
tggagcagttggcgta

SEQ ID NO: 13 - RNA, 16 nt, synthetic construct
  source 1..16: mol_type = other RNA
tggagcggttggcgta

SEQ ID NO: 14 - RNA, 16 nt, synthetic construct
  source 1..16: mol_type = other RNA
tggagctgtcggcgta

SEQ ID NO: 15 - RNA, 16 nt, synthetic construct
  source 1..16: mol_type = other RNA
tggagctgtaggcgta

SEQ ID NO: 16 - RNA, 16 nt, synthetic construct
  source 1..16: mol_type = other RNA
tggagctgtgggcgta

SEQ ID NO: 17 - RNA, 60 nt, synthetic construct
  source 1..60: mol_type = other RNA
ctgaatataaacttgtggtagttggagctgttggcgtaggcaagagtgccttgacgatac

SEQ ID NO: 18 - DNA, 30 bp, synthetic construct
  source 1..30: mol_type = other DNA
  modified_base 1: mod_base = OTHER; note = Locked nucleic acid
  modified_base 30: mod_base = OTHER; note = Locked nucleic acid
  modified_base 22: mod_base = OTHER; note = 2'-O-methoxyethyl
  modified_base 2: mod_base = OTHER; note = 2'-O-Methyladenosine
  modified_base 3: mod_base = cm
  modified_base 4: mod_base = gm
  modified_base 15: mod_base = cm
  modified_base 16: mod_base = um
  modified_base 27: mod_base = um
  modified_base 28..29: mod_base = cm
  misc_feature 1..15: note = DNA
  misc_feature 17..26: note = DNA
  misc_feature 28..30: note = DNA
  misc_feature 16: note = RNA
  misc_feature 27: note = RNA
tacgccaaggccatctacaacgaagctcca

SEQ ID NO: 19 - RNA, 21 nt, synthetic construct
  source 1..21: mol_type = other RNA
tgttggagctgtggcgtaggc

SEQ ID NO: 20 - DNA, 22 bp, synthetic construct
  source 1..22: mol_type = other DNA
gcctacgccaacagctccaact

SEQ ID NO: 21 - DNA, 60 bp, synthetic construct
  source 1..60: mol_type = other DNA
ctgaatataaacttgtggtagttggagctggtggcgtaggcaagagtgccttgacgatac

SEQ ID NO: 22 - DNA, 18 bp, synthetic construct
  source 1..18: mol_type = other DNA
haggctagctacaacgaa

SEQ ID NO: 23 - RNA, 16 nt, synthetic construct
  source 1..16: mol_type = other RNA
tggagctggtggcgta

SEQ ID NO: 24 - DNA, 16 bp, synthetic construct
  source 1..16: mol_type = other DNA
tggacctggtggcgta

SEQ ID NO: 25 - DNA, 16 bp, synthetic construct
  source 1..16: mol_type = other DNA
tacgccaccaggtcca

SEQ ID NO: 26 - DNA, 16 bp, synthetic construct
  source 1..16: mol_type = other DNA
tggacctgttggcgta

SEQ ID NO: 27 - DNA, 16 bp, synthetic construct
  source 1..16: mol_type = other DNA
tacgccaacaggtcca

SEQ ID NO: 28 - DNA, 30 bp, synthetic construct; identical to SEQ ID NO: 2
  source 1..30: mol_type = other DNA

SEQ ID NO: 29 - DNA, 30 bp, synthetic construct; identical to SEQ ID NO: 2
  source 1..30: mol_type = other DNA

SEQ ID NO: 30 - DNA, 30 bp, synthetic construct; identical to SEQ ID NO: 2
  source 1..30: mol_type = other DNA
  modified_base 1..3: mod_base = OTHER; note = Locked nucleic acid
  modified_base 28..30: mod_base = OTHER; note = Locked nucleic acid
  modified_base 22: mod_base = gm

SEQ ID NO: 31 - DNA, 30 bp, synthetic construct; identical to SEQ ID NO: 2
  source 1..30: mol_type = other DNA
  misc_feature 1..26: note = DNA
  misc_feature 27: note = RNA
  misc_feature 28..30: note = DNA
  modified_base 2: mod_base = OTHER; note = 2'-O-Methyladenosine
  modified_base 3: mod_base = cm
  modified_base 4: mod_base = gm
  modified_base 22: mod_base = gm
  modified_base 27: mod_base = um
  modified_base 28..29: mod_base = cm
  modified_base 1: mod_base = OTHER; note = Locked nucleic acid
  modified_base 30: mod_base = OTHER; note = Locked nucleic acid

SEQ ID NO: 32 - DNA, 30 bp, synthetic construct; identical to SEQ ID NO: 2
  source 1..30: mol_type = other DNA
  modified_base 15: mod_base = cm
  modified_base 16: mod_base = gm
  modified_base 22: mod_base = gm

SEQ ID NO: 33 - DNA, 30 bp, synthetic construct; identical to SEQ ID NO: 2
  source 1..30: mol_type = other DNA
  modified_base 15..16: mod_base = OTHER; note = 2'-O-methoxyethyl
  modified_base 22: mod_base = OTHER; note = 2'-O-methoxyethyl

SEQ ID NO: 34 - DNA, 30 bp, synthetic construct; identical to SEQ ID NO: 2
  source 1..30: mol_type = other DNA
  modified_base 15: mod_base = cm
  modified_base 16: mod_base = tm
  modified_base 22: mod_base = OTHER; note = 2'-O-methoxyethyl

SEQ ID NO: 35 - DNA, 30 bp, synthetic construct; identical to SEQ ID NO: 2
  source 1..30: mol_type = other DNA
  misc_feature 1..26: note = DNA
  misc_feature 28..30: note = DNA
  misc_feature 27: note = RNA
  modified_base 1: mod_base = OTHER; note = Locked nucleic acid
  modified_base 30: mod_base = OTHER; note = Locked nucleic acid
  modified_base 2: mod_base = OTHER; note = 2'-O-Methyladenosine
  modified_base 3: mod_base = cm
  modified_base 4: mod_base = gm
  modified_base 15: mod_base = cm
  modified_base 16: mod_base = tm
  modified_base 22: mod_base = OTHER; note = 2'-O-methoxyethyl
  modified_base 27: mod_base = um
  modified_base 28..29: mod_base = cm

SEQ ID NO: 36 - DNA, 30 bp, synthetic construct; identical to SEQ ID NO: 2
  source 1..30: mol_type = other DNA
  modified_base 22: mod_base = gm

SEQ ID NO: 37 - DNA, 30 bp, synthetic construct; identical to SEQ ID NO: 2
  source 1..30: mol_type = other DNA
  modified_base 22: mod_base = OTHER; note = 2'-O-methoxyethyl

SEQ ID NO: 38 - DNA, 30 bp, synthetic construct; identical to SEQ ID NO: 2
  source 1..30: mol_type = other DNA
  misc_feature 1..26: note = DNA
  misc_feature 28..30: note = DNA
  modified_base 1: mod_base = OTHER; note = Locked nucleic acid
  modified_base 30: mod_base = OTHER; note = Locked nucleic acid
  modified_base 2: mod_base = OTHER; note = 2'-O-Methyladenosine
  modified_base 3: mod_base = cm
  modified_base 4: mod_base = gm
  modified_base 27: mod_base = um
  misc_feature 27: note = RNA
  modified_base 28..29: mod_base = cm

SEQ ID NO: 39 - DNA, 30 bp, synthetic construct; identical to SEQ ID NO: 2
  source 1..30: mol_type = other DNA
  modified_base 15: mod_base = cm
  modified_base 16: mod_base = um
  modified_base 22: mod_base = gm

SEQ ID NO: 40 - DNA, 30 bp, synthetic construct; identical to SEQ ID NO: 2
  source 1..30: mol_type = other DNA
  modified_base 1: mod_base = OTHER; note = Locked nucleic acid
  modified_base 30: mod_base = OTHER; note = Locked nucleic acid
  misc_feature 1..15: note = DNA
  misc_feature 16: note = RNA
  misc_feature 17..26: note = RNA
  misc_feature 27: note = RNA
  misc_feature 28..30: note = DNA
  modified_base 2: mod_base = OTHER; note = 2'-O-Methyladenosine
  modified_base 3: mod_base = cm
  modified_base 4: mod_base = gm
  modified_base 15: mod_base = cm
  modified_base 16: mod_base = um
  modified_base 27: mod_base = um
  modified_base 28..29: mod_base = cm

SEQ ID NO: 41 - DNA, 30 bp, synthetic construct; identical to SEQ ID NO: 2
  source 1..30: mol_type = other DNA
  modified_base 1: mod_base = um
  modified_base 2: mod_base = OTHER; note = 2'-O-Methyladenosine
  modified_base 3: mod_base = cm
  modified_base 4: mod_base = gm
  modified_base 5: mod_base = cm
  modified_base 10: mod_base = gm
  modified_base 15: mod_base = cm
  modified_base 16: mod_base = um
  modified_base 19: mod_base = OTHER; note = 2'-O-Methyladenosine
  modified_base 22: mod_base = gm
  modified_base 23: mod_base = OTHER; note = 2'-O-Methyladenosine
  modified_base 27: mod_base = um
  modified_base 28..29: mod_base = cm
  modified_base 30: mod_base = OTHER; note = 2'-O-Methyladenosine
  misc_feature 1: note = RNA
  misc_feature 2..15: note = DNA
  misc_feature 16: note = RNA
  misc_feature 17..26: note = DNA
  misc_feature 27: note = RNA
  misc_feature 28..30: note = DNA

SEQ ID NO: 42 - DNA, 30 bp, synthetic construct; identical to SEQ ID NO: 2
  source 1..30: mol_type = other DNA
  modified_base 1: mod_base = um
  modified_base 2: mod_base = OTHER; note = 2'-O-Methyladenosine
  modified_base 3: mod_base = cm
  modified_base 4: mod_base = gm
  modified_base 5: mod_base = cm
  modified_base 10: mod_base = gm
  modified_base 15: mod_base = cm
  modified_base 16: mod_base = um
  modified_base 19: mod_base = OTHER; note = 2'-O-Methyladenosine
  modified_base 22: mod_base = gm
  modified_base 23: mod_base = OTHER; note = 2'-O-Methyladenosine
  modified_base 27: mod_base = um
  modified_base 28..29: mod_base = cm
  modified_base 30: mod_base = OTHER; note = 2'-O-Methyladenosine
  misc_feature 1: note = RNA
  misc_feature 2..15: note = DNA
  misc_feature 16: note = RNA
  misc_feature 17..26: note = DNA
  misc_feature 27: note = RNA
  misc_feature 28..30: note = DNA

SEQ ID NO: 43 - RNA, 60 nt, synthetic construct
  source 1..60: mol_type = other RNA
ctgaatataaacttgtggtagttggagcagttggcgtaggcaagagtgccttgacgatac

SEQ ID NO: 44 - DNA, 21 bp, synthetic construct
  source 1..21: mol_type = other DNA
tcctcttgacctgctgtgtcg

SEQ ID NO: 45 - DNA, 45 bp, synthetic construct
  source 1..45: mol_type = other DNA
ttattataaataatgactgaatataaacttgtggtagttggacct

SEQ ID NO: 46 - DNA, 26 bp, synthetic construct
  source 1..26: mol_type = other DNA
accatcttccaggagcgagatccctc

SEQ ID NO: 47 - DNA, 27 bp, synthetic construct
  source 1..27: mol_type = other DNA
tgcaggaggcattgctgatgatcttga